(19) United States
(12) Patent Application Publication
(10) Pub. No.: US 2026/0365976 A1
(43) Pub. Date: Nov. 19, 2026

(54) ENHANCED GAMMA DELTA T CELLS FOR IMMUNOTHERAPY

(71) Applicant: The Regents of the University of California, Oakland, CA (US)

(72) Inventors: Lili Yang, Los Angeles, CA (US); Derek Lee, Los Angeles, CA (US); Zachary Spencer Dunn, South Pasadena, CA (US)

(73) Assignee: The Regents of the University of California, Oakland, CA (US)

(21) Appl. No.: 19/130,270

(22) PCT Filed: Dec. 15, 2023
(86) PCT No.: PCT/US2023/084241

§ 371 Date: May 15, 2025

Related U.S. Application Data
(60) Provisional application No. 63/432,814, filed on Dec. 15, 2022.
(60) Provisional application No. 63/479,278, filed on Jan. 10, 2023.
(60) Provisional application No. 63/502,881, filed on May 17, 2023.

(51) Int. Cl. C12N 5/0783; C12N 5/00

(52) U.S. Cl. CPC C12N 5/0636; C12N 5/0018; C12N 2500/42; C12N 2500/99; C12N 2501/2302; C12N 2501/2307; C12N 2501/2315; C12N 2501/2321; C12N 2501/515; C12N 2501/727; C12N 2510/00

(57) ABSTRACT

Aspects of the present disclosure relate to methods and compositions related to the selection and expansion of immune cells, including T cells expressing CD¹⁶^{^{Hi}} in combination with a Vδ2 T cell receptor. The T cells generated by the methods disclosed herein are suitable for allogeneic cellular therapy because they do not induce graft-versus-host disease (GvHD) and resist host immune allorejection. Consequently, such cells are suitable for off-the-shelf use in clinical therapy for diseases such as cancers, infectious diseases, autoimmune diseases, cardiac diseases, and neuronal diseases.

WO

### CROSS REFERENCE TO RELATED APPLICATIONS

This application claims the benefit under 35 U.S.C. Section 119(e) of co-pending and commonly-assigned U.S. Provisional Patent Application No. 63/432,814, filed Dec. 15, 2022, U.S. Provisional Patent Application No. 63/479,278, filed Jan. 10, 2023, and U.S. Provisional Patent Application No. 63/502,881, filed May 17, 2023, all entitled “ENHANCED GAMMA DELTA T CELLS FOR IMMUNOTHERAPY”, which applications are incorporated by reference herein.

### TECHNICAL FIELD

Embodiments of the disclosure concern at least the fields of immunology, cell biology, molecular biology, and medicine.

### BACKGROUND OF THE INVENTION

Gamma delta (γδ) T cells are a small subpopulation of T lymphocytes having the ability to bridge innate and adaptive immunity. The majority of γδ T cells in adult human blood exhibit Vγ9Vδ2 T cell receptors and respond to small phosphorylated nonpeptide antigens, called phosphoantigens (pAgs), which are commonly produced by malignant cells (see, e.g., Yang et al., Immunity 50, 1043-1053.e5 (2019)). Unlike conventional αβ T cells, γδ T cells do not recognize polymorphic classical major histocompatibility complex (MHC) molecules and are therefore free of graft versus host disease (GvHD) risk when adoptively transferred into an allogeneic host. Additionally, γδ T cells have several other unique features that make them ideal cellular carriers for developing off-the-shelf cellular therapy for cancer. These features include: 1) γδ T cells have roles in cancer immunosurveillance; 2) γδ T cells have the remarkable capacity to target tumors independent of tumor antigen-and major histocompatibility complex (MHC)-restrictions; 3) γδ T cells can employ multiple mechanisms to attack tumor cells through direct killing and adjuvant effects; and 4) γδ T cells can express a surface receptor, FcγRIII (CD16), that is involved in antibody-dependent cellular cytotoxicity (ADCC) and can be potentially combined with monoclonal antibody for cancer therapy.

Unfortunately, however, the development of an allogeneic off-the-shelf γδ T cellular product is greatly hindered by their availability-these cells are of low number and high variability in humans (~1-5% T cells in human blood), making it difficult to produce therapeutic numbers of γδ T cells using blood cells from allogeneic human donors. Furthermore, γδ T cells are a heterogeneous cell population with different transcriptional programming, phenotype, and functionality, and there is inter-donor variability of γδ T cells profiles. Certain subsets and donor-specific attributes of γδ T cells may be desirable for cancer adoptive cell immunotherapy (ACT), and thus it is of importance to identify biomarkers indicative of greater potency.

There is a need in the art for means for generating large quantities of γδ T cells with potent effector capabilities. Specifically, novel methods and materials that can reliably generate large quantities of yo T cells are pivotal to developing off-the-shelf γδ T cell therapies that are useful in the treatment of a wide variety of pathological conditions.

### SUMMARY OF THE INVENTION

Cancer remains a leading cause of death worldwide and in the United States. Chimeric alpha beta (αβ) antigen receptor-T (CAR-T) cells have transformed the treatment of hematological malignancies but have not had the same success in combatting solid tumors. Furthermore, all the FDA approved CAR-T cell therapies are autologous, which is a critical bottleneck in their scalability, affordability, and accessibility, and can cause severe adverse events. To create allogeneic, “off-the shelf” cell therapies that are both safe and effective for the treatment of both liquid and solid tumor types, we have focused on gamma delta (γδ) T cells as a cell source for genetic engineering and cancer treatments, and in particular, the Vδ2 subset of γδ T cells, a T cell population that does not cause graft versus-host-disease and which further possesses intrinsic cancer killing abilities. Unfortunately, however, their clinical application for cancer immunotherapy has been limited by their scarcity and persistence, as well as by tumor immunosuppression mechanisms. In this context, the ability to manufacture a therapeutic γδ T cell population or a cell population that can be used to create a therapeutic γδ T cell population “off-the-shelf” increases the availability and usefulness of new cellular therapies.

As disclosed herein, we have invented a unique γδ T cell platform designed to address the need for new methods and material useful in cellular therapies, more particularly, to address the need for cellular therapeutics that are not hampered by the challenges posed in individualizing therapy using autologous cells. As discussed below, using selected CD16 high (CD16^{Hi}) Vδ2 T cell screening and expansion methods as disclosed herein, we overcome a number of limitations associated with conventional reagents and methods used in T lymphocyte growth and expansion methodologies. As disclosed herein, we screened donors for CD16 expression on Vδ2 T cells in order to focus on CD16 as a biomarker for Vδ2 T cell donor selection. Our studies discovered that CD16^{Hi} Vδ2 T cells exhibit more robust cytotoxicity activity than CD16 low (CD16^{Lo}) Vδ2 T cells, and for example perform well in antibody-dependent cellular cytotoxicity assays. Furthermore, CD16^{Hi} Vδ2 T cells were found to display a gene profile reduced for Th17 function. Building upon our discoveries, we developed a Vδ2 T lymphocyte expansion method and associated media materials that employ a combination of a γδ T cell stimulator (e.g., the bisphosphonate zoledronate), cytokines, and GSK-3β inhibitors to achieve upwards of 10,000-fold expansion rates of CD16^{Hi} Vδ2 T cells, expansion rates which are about 10 to 20-fold greater than expansion rates observed with conventional approaches. The Vδ2 T cells made by embodiments of the invention can be expanded for over one month and restimulated while maintaining a highly desirable less differentiated memory status. Moreover, the resulting memory-like Vδ2 T cells outperformed Vδ2 T cells expanded by conventional approaches in a long-term stress assay. These discoveries and associated advancements in Vδ2 T cell technologies can be harnessed to generate potent, high quality Vδ2 cells at a scale suitable for widespread clinical applications.

3

The invention disclosed herein has a number of embodiments. Embodiments of the invention include methods of growing mammalian cells (typically human T lymphocytes), the methods comprising: obtaining a population of lymphocytes (e.g., from peripheral blood of one or more donors); identifying donors based on the V82 T cell CD16 expression; selecting and/or purifying donor cells within the population of T lymphocytes that express CD16^{Hi} in combination with Vδ2; and then expanding the selected and/or purified T lymphocytes that express CD16 in combination with Vδ2. In certain embodiments of the invention, the cells are expanded by 1,000, 2,000, 3,000, 4,000, 5,000, 6,000, 7,000, 8,000, 9,000, or 10,000 fold. In some embodiments, the expansion period lasts over 3, 4, 5 or 6 weeks. In some embodiments, the cells are restimulated during the expansion period. Typically in such methods, the selected and/or purified T lymphocytes that express CD16 in combination with Vδ2 are expanded by disposing the T lymphocytes in a media comprising a γδ T cell stimulator (e.g., the bisphosphonate zoledronate), a combination of cytokines (e.g. IL-2, IL-15,IL-12, IL-18, IL-7, IL-21, TGF-β), a Wnt activator (e.g. Wnt3a) and/or a GSK-3β inhibitor (e.g. TWS119). In certain embodiments of the invention, the method includes testing/examining cells within the population of CD16^{Hi} Vδ2 lymphocytes in assays of cellular cytotoxicity such as antibody-dependent cellular cytotoxicity assays.

Embodiments of the invention include a cell culture media for expanding T lymphocytes that express CD16 in combination with Vδ2, the media comprising: a γδ T cell stimulator (e.g., a bisphosphonate), a combination of cytokines, a Wnt activator and/or a GSK-3β inhibitor. In certain embodiments of the invention, this media further includes a population of T cells that have been selected for T lymphocytes that express CD16 in combination with Vδ2. Optionally, this population of T cells has been selected or enriched using an antibody that specifically binds CD16, an antibody that specifically binds Vδ2; magnetic bead sorting; and/or fluorescent activated cell sorting (FACS). Related embodiments of the invention also include methods of making a cell culture media for expanding T lymphocytes that express CD16 in combination with Vδ2, the methods comprising combining together a γδ T cell stimulator (e.g., a bisphosphonate), a combination of cytokines, a Wnt activator and/or a GSK-31 inhibitor so as to form the cell culture media. In some embodiments of the invention, tyrosine kinase inhibitors are included in the cell culture media. Certain embodiments of this invention include further disposing T cells selected to express CD16 in combination with Vδ2 into this cell culture media. In certain embodiments of the invention, the population of T cells has been modified to modulate the expression of one or more endogenous genes (e.g., a human leukocyte antigen gene or the like). In some embodiments of the invention, the population of cells is engineered to express an exogenous transgene (e.g., a chimeric alpha beta (αβ) antigen receptor transgene).

Embodiments of the invention also include methods of treating patients with a CD16^{Hi} Vδ2 T cell or cell population as disclosed herein. Such embodiments of the invention include methods of treating a subject in need of gamma delta T cells (e.g., to fight a disease such as an autoimmune disease or a cancer or an infection such as viral, bacterial or parasitic infection) which comprises administering to the subject functional CD16^{Hi} Vδ2 gamma delta T cells selected to target the appropriate pathology.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1: CD16 serves as a biomarker to screen peripheral blood mononuclear cell (PBMC) donors for high performance Vδ2 T cells. a Experimental design to generate PBMC-derived Vγ9Vδ2 T (referred to as Vδ2 T) cells. Zoledronate (ZOL) and IL-2 were used to activate and expand Vδ2 T cells. b Pie chart showing the proportions of CD16 high (CD16^{Hi}) and CD16 low (CD16^{Lo}) Vδ2 T cell donors. Note, a total of 30 healthy donors were screened. The cutoff between CD16^{Hi} and CD16^{Lo} donors was 35% of CD16⁻ cells out of total Vδ2 T cells. c FACS quantification of %CD16⁺ cells of total Vδ2 T cells before and after activation and expansion. PBMCs from 30 different donors were used. Note, Vδ2 T cells were expanded for 10~14 days. d Representative FACS plots of (c). e Expansion of CD16^{Hi} and CD16^{Lo} Vδ2 T cells over 14 days (n=5; n indicates different donors). f-h Studying the in vitro antitumor efficacy of CD16^{Hi} and CD16^{Lo} Vδ2 T cells in the presence or absence of ZOL. Two human ovarian cancer cell lines were studied as the tumor targets: OVCAR3-FG and SKOV3-FG; both cancer cell lines were engineered to express the firefly luciferase and green fluorescence protein (FG) dual reporters. f Experimental design. g Tumor cell killing data at 24 h (n=3 from 3 different donors). h ELISA measurements of IFN-γ production (left), and FACS measurements of intracellular perforin (middle) and granzyme B (right) production, at 24 h (E: T ratio=1:1; n=3 from 3 different donors). i-k Studying the overexpression of CD16 in CD16^{Lo} Vδ2 T cells. CD16^{Lo} Vδ2 T cells were transduced with a lentivector encoding the human CD16 gene (Lenti/CD16). i Schematic design of overexpression experiment. j FACS detection of CD16 overexpression on CD161^{Lo} Vδ2 T cells transduced with titrated amounts of Lenti-CD16. k In vitro killing of OVCAR3-FG cells by Lenti/CD16 transduced CD16^{Lo} Vδ2 T cells in the presence of ZOL at 24 h after co-culture (n=3). Representative of >10 (a-d), 5 (e), 3 (f-h), and 1 (i-k) experiments. Data are presented as the mean ±SEM. ns, not significant; *p<0.05; **p<0.01; ***p<0.001; ****p<0.0001 by Student's t test (e, g) or by one-way ANOVA (h).

FIG. 2: CD16^{Hi} Vδ2 T cells display enhanced cytotoxic gene signatures. Vδ2 T cells expanded from 13 PBMC donors were subject to RNA-Seq analysis. a Dot plot showing the CD16 mRNA expression in Vδ2 T cells (CPM: counts per million reads mapped). The samples were categorized into CD16^{Hi} (n=3) and CD16^{Lo} (n=10) Vδ2 T cells using k-means clustering algorithm (k=2). b Principal-component analysis (PCA) plot showing the coordination of CD16^{Hi} and CD16^{Lo} Vδ2 T cells. c Volcano plot showing the differential gene expression between CD16^{Hi} and CD16^{Lo} Vδ2 T cells. FC, fold change. NS, not significant. d Gene set enrichment analysis (GSEA) plots showing a significant enrichment of indicated gene signatures in CD16^{Hi} Vδ2 T cells. NES, normalized enrichment score. e Cnet plot showing the enriched GO pathways. f Correlation plot showing the relationship between CD16 expression in Vδ2 T cell samples and immune cell type scores. Circle size and color intensity are proportional to correlation; red and blue represent positive and negative correlations, respectively. Representative of 1 experiment.

FIG. 3: CD16^{Hi} Vδ2 T cells can be engineered with CAR/IL-15 while retaining expansion capacity and memory status. a Experimental design to generate the MCAR/IL-15 engineered CD16^{Hi} Vδ2 T (MCAR15-Vδ2T) cells. Note, ZOL and IL-2 were used to activate and expand Vδ2 T cells. CAR, chimeric antigen receptor. b Schematic of the indicated lentivectors. Lenti/MCAR, lentivector encoding a mesothelin (MSLN)-targeting CAR (MCAR); Lenti/MCAR15: lentivector encoding the same MCAR as well as a secreting form of human IL-15. scFv: single-chain variable fragment; V_{H}: variable heavy chain; V_{L}: variable light chain; H: CD8 hinge; TM: CD28 transmembrane domain; CD28: CD28 intracellular domain; CD3ζ: CD3ζ (intracellular domain; IL-15: interleukin 15. c FACS analysis of CAR expression on CD16^{Hi} Vδ2 T cells transduced with mock (denoted as non-transduced Vδ2 T cells; NT-Vδ2T cells), or with Lenti/MCAR (denoted as MCAR-Vδ2T cells), or with Lenti/MCAR15 (denoted as MCAR15-Vδ2T cells). d Quantification of c (n=10; n indicates different donors). e Expansion of the indicated Vδ2 T cells over 14 days of culture (n=5; n indicates different donors). f FACS analysis of the cellular composition of expanded MCAR15-Vδ2T cells. g Quantification of f (n=10; n indicates different donors), NK cells were gated as CD45⁺CD56⁺CD3⁻ cells. h FACS detection of IL-15 signaling events in MCAR15-Vδ2T cells. Phosphorylation of STAT5 (pSTAT5) and the upregulation of anti-apoptotic transcription factors Bcl-XL and BcL-2 were studied. Cells were cultured in cytokine free media for 48 h before intracellular staining. i ELISA measurements of IL-15 production from MCAR15-Vδ2T cells in the presence or absence of human ovarian cancer cell line OVCAR3-FG for 48 h (n=3). j-l Studying the memory phenotype of MCAR15-Vδ2T cells. j Schematic plot defining memory T cell subsets by the cell surface expression of CD27 and CD45RA biomarkers. Stem cell-like memory (T_{SCM}): CD27⁺CD45RA⁺; central memory (T_{CM}): CD27⁺CD45RA⁻; effector memory (T_{EM}): CD27⁻CD45RA⁻; terminally differentiated effector memory (T_{EMRA}): CD27⁻CD45RA⁺. k FACS detection of CD27 and CD45RA expression on CAR15⁺ and CAR15⁻MCAR15-Vδ2T cells. I Quantification of k (n=5; n indicates different donors). Representative of 1 (b), 3 (h, i), 5 (j-l), and >10 (c-g) experiments. Data are presented as the mean ±SEM. ns, not significant; *p<0.05; ****p<0.0001 by Student's t test (d, l) or by one-way ANOVA (e, i).

FIG. 4: MCAR15-Vδ2T cells can effectively kill tumor cells via CAR/TCR dual-targeting mechanisms. Three human ovarian cancer cell lines (OVCAR3-FG, OVCAR8-FG, and SKOV3-FG) and four effector cells (Vδ2T, MCAR-Vδ2T, MCAR15-Vδ2T, and MCAR-T) were used in the study. MCAR-T indicates conventional αβ T cells engineered to express the same MCAR (as a benchmark control). The same CD16^{Hi} donor PBMCs were used to generate all 4 types of effector cells. a-e In vitro 24-hour tumor cell killing assay. a Experimental design. Tumor cells and effector cells were co-cultured for 24 hours, then were subject to analysis. b FACS plots showing the expression of mesothelin (MSLN) tumor antigen on the indicated ovarian cancer cell lines. c Tumor cell killing data collected at 24 h (n=3). d FACS detection of perforin and granzyme B intracellular production by MCAR15-Vδ2T cells at 24 h (E:T ratio=1:1). e ELISA measurements of IFN-γ production at 24 h (E:T ratio=1:1; n=3). f-i In vitro repeated tumor cell challenge assay. f Experimental design. Effector cells were mixed with tumor cells and rechallenged every 3 days, and the tumor cell killing data were collected at 24 h after every dosing of tumor cells (E:T ratio=2:1; n=3). g Tumor cell killing data collected over time. h Generation and FACS validation of an MSLN-knockout OVCAR3-FG (^{KO}OVCAR3-FG) cell line. i ^{KO}OVCAR 3-FG tumor cell killing data collected after the 3^{rd} tumor cell rechallenge. Representative of 3 (a, c-g, i), and 1 (b, h) experiments. Data are presented as the mean±SEM. ns, not significant; *p<0.05; **p<0.01; ***p<0.001; ****p<0.0001 by Student's t test (c, e) or by one-way ANOVA (i).

FIG. 5: MCAR15-Vδ2T cells can also effectively target tumor cells via an antibody-dependent cell-mediated cytotoxicity (ADCC) mechanism. a-g In vitro ADCC assay. Three human ovarian cancer cell lines (OVCAR3-FG, OVCAR8-FG, and SKOV3-FG) and four effector cells (Vδ2T, MCAR-Vδ2T, MCAR15-Vδ2T, and MCAR-T as a benchmark control) were included in the study. The same CD16^{Hi} donor PBMCs were used to generate all 4 types of effector cells. a Experimental design. Data were collected at 24 h after co-culture. Anti-HER2 Ab: monoclonal antibody trastuzumab. b FACS detection of HER2 expression on the indicated tumor cell lines. c Killing of OVCAR3-FG tumor cells by Vδ2T cells in the presence of titrated amounts of either isotype control or anti-HER2 Ab (n=3). d-f Tumor cell killing data of (d) OVCAR3-FG, (e) OVCAR8-FG, and (f) SKOV3-FG in the presence or absence of anti-HER2 Ab (anti-HER2 Ab concentration=0.1 μg/mL; E:T ratio=0.5:1; n=3). g ELISA measurements of IFN-γ production at 24 h in the presence or absence of anti-HER 2 Ab (anti-HER2 Ab concentration=0.1 μg/mL; E:T ratio=1:1; n=3). h, i In vitro repeated tumor cell challenge assay studying ADCC. h Experimental design. Effector cells were mixed with tumor cells and rechallenged every 3 days. Tumor cell killing data was measured at 24 h post co-culture with or without the addition of anti-HER2 antibody (anti-HER2 Ab concentration=0.1 μg/ml; E:T ratio=2:1; n=3). i ^{KO}OVCAR3-FG tumor cell killing data collected over time. Representative of 3 experiments. Data are presented as the mean±SEM. ns, not significant; **p<0.01; ***p<0.001; ****p<0.0001 by Student's t test (c) or by one-way ANOVA (d-g).

FIG. 6: In vivo antitumor efficacy and safety of MCAR15-Vδ2T cells in an intraperitoneal tumor model. OVCAR3-FG human ovarian cancer cells were injected i.p. into NSG mice on Day 0, followed by i.p. administration of effector cells on day 14 (n=5 mice per group). Four experimental groups were included: Vehicle (mice receiving no effector cells), MCAR-T (mice receiving MCAR-T cells), MCAR-Vδ2T (mice receiving MCAR-Vδ2T cells), and MCAR15-Vδ2T (mice receiving MCAR15-Vδ2T cells). Note, the same CD16^{Hi} donor PBMCs were used to generate all 3 types of effector cells. a Experimental design. b BLI images showing tumor loads in experimental mice over time. c Quantification of b. TBL, total body luminescence. d Tumor loads on day 55. e Kaplan-Meier survival curves of experimental mice over time. Representative of 3 experiments. Data are presented as the mean±SEM. ns, not significant; *p<0.05; **p<0.01; ****p<0.0001 by one-way ANOVA (d), or by log rank (Mantel-Cox) test adjusted for multiple comparisons (e).

FIG. 7: In vivo antitumor efficacy and safety of MCAR15-Vδ2T cells in a subcutaneous tumor model. OVCAR8 human ovarian cancer cells were injected s.c. into NSG mice on day 0, followed by intravenous (i.v.). administration of effector cells on Day 7. Four experimental groups were included: Vehicle (mice receiving no effector cells), MCAR-T (mice receiving MCAR-T cells), MCAR-Vδ2T (mice receiving MCAR-Vδ2T cells), and MCAR15-Vδ2T (mice receiving MCAR15-Vδ2T cells). Note, the same CD16^{Hi} donor PBMCs were used to generate all 3 types of effector cells. a Experimental design. b Tumor growth over time. c Tumor size measurements collected on day 57 (n=5). d FACS quantification of MCAR-Vδ2T and MCAR15-Vδ2T cells across tissues of experimental mice collected on day 57 (n=5). e H&E-stained tissue sections collected on day 57. Scale bar, 100 μm. Representative of 3 experiments. Data are presented as the mean±SEM. ns, not significant; ****p<0.0001 by one-way ANOVA (c).

FIG. 8: Characterization of the CD16^{+/-}Vδ2 T cells generated from the CD16^{Hi} or CD16^{Lo} PBMC donors. Vδ2 T cells were generated from the CD16^{Hi} or CD16^{Lo} donor PBMCs following the experimental design shown in FIG. 1a, then were subject to FACS analysis for a, b the gating strategy to select CD16⁺ and CD16⁻ Vδ2 T cells from CD16^{Hi} or CD16^{Lo} donors, c, d surface expression markers (n=5), and e-h intracellular perforin and granzyme B production at 24 h after tumor co-culture (E:T ratio=1:1; n=3 from 3 different donors). CD16⁺ and CD16⁻ Vδ2 T cells were gated based on cell surface CD16 expression. Representative of >10 (a, b), 2 (c, d), and 3 (e-h) experiments. Data are presented as the mean±SEM. ns, not significant; *p<0.05; **p<0.01; ****p<0.0001 by Student's t test (c, d) or by one-way ANOVA (e-h).

FIG. 9: Transcriptome characterization of Vδ2 T cells in relation to CD16 expression. a Heatmaps showing the expression levels of the selected genes in Vδ2 T cells generated from three CD16^{Hi} PBMC donors and ten CD16^{Lo} PBMC donors. b Gene set enrichment analysis showing the relationship between CD16 expression and biological process pathways in Vδ2 T cells generated from all 13 PBMC donors (including the three CD16^{Hi} donors and ten CD16^{Lo} donors). The top 30 most significantly activated or suppressed biological process pathways are shown. Representative of 1 experiment.

FIG. 10: Comparing the in vitro antitumor efficacy of CD16^{Hi} and CD16^{Lo} MCAR15-Vδ2T cells. Vδ2 T cells were cultured from the CD16^{Hi} or CD16^{Lo} donor PBMCs and were engineered to express MCAR and IL-15. The resulting cell products are denoted as ^{16H}MCAR15-Vδ2T or ^{16H}MCAR15-Vδ2T cells, respectively. a, b In vitro tumor cell killing assay. An OVCAR3-FG human ovarian cancer cell line was used. a Experimental design. b Tumor cell killing data collected as 24 h after co-culture (n=3; n indicates different donors). c, d In vitro antibody-dependent cell-mediated cytotoxicity (ADCC) assay. A SKOV3-FG human ovarian cancer cell line was used. c Experimental design. d Tumor cell killing data collected at 24 h after co-culture (Anti-HER2 Ab concentration=0.1 μg/mL; n=3; n indicates different donors). Representative of 3 experiments. Data are presented as the mean±SEM. ns, not significant; *p<0.05; ***p<0.001; ****p<0.0001 by Student's t test (b, d).

FIG. 11: FMCAR15-Vδ2T cells can target M2-polarized human macrophages. a Experimental design to generate human monocyte-derived macrophages (MDM), either non-polarized (M0) or M2-polarized. M-CSF, macrophage colony-stimulating factor; Mo, macrophage. b FACS detection of CD11b and CD14 expression on M2-polarized macrophages. c FACS detection of M2 macrophage markers (i.e., CD163 and CD206) on M2-polarized macrophages. d, e Studying the in vitro killing of M2-polarized macrophages by MCAR15-Vδ2T effector cells. d Experimental design. e Data collected at 24 h after co-culture (E: Mφ ratio=1:1, n=3). Data are presented as the mean±SEM. ns, not significant; *p<0.05; ***p<0.001; ****p<0.0001 by one-way ANOVA (e).

FIG. 12: Comparing the in vivo antitumor efficacy of CD16^{Hi} and CD16^{Lo} MCAR15-Vδ2T cells. Vδ2 T cells were cultured from the CD16^{Hi} or CD16^{Lo} donor PBMCs and were engineered to express MCAR and IL-15. The resulting cell products are denoted as ^{16H}MCAR15-Vδ2T or ^{16H}MCAR15-Vδ2T cells, respectively. a Experimental design. Three experimental groups were included: Vehicle (mice receiving no effector cells; n=5), ^{16L}MCAR15-Vδ2T (mice receiving ^{16H}MCAR15-Vδ2T cells; n=9 from 3 different donors), and ^{16L}MCAR15-Vδ2T (mice receiving ^{16L}MCAR 15-Vδ2T cells; n=9 from 3 different donors). b Tumor growth over time. c Tumor sizes measured on day 57. Representative of 2 experiments. Data are presented as the mean±SEM. **p<0.01; ****p<0.0001 by one-way ANOVA (c).

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention.

It is specifically noted that any embodiment discussed herein in the context of a particular cell or cell population embodiment may be employed with respect to any other cell or cell population embodiment. Moreover, any embodiment employed in the context of a specific method may be implemented in the context of any other methods described herein. Furthermore, aspects of different methods described herein may be combined so as to achieve other methods, as well as to create or describe the use of any cells or cell populations. It is specifically contemplated that aspects of one or more embodiments may be combined with aspects of one or more other embodiments described herein. Furthermore, any method described herein may be phrased to set forth one or more uses of cells or cell populations described herein. For instance, use of γδ T cells or a γδ T cell population can be set forth from any method described herein.

As noted above, there is a need in the art for methods and materials that can reliably generate potent γδ T cells in large quantities. These technologies are pivotal to developing off-the-shelf T cell therapies. Such methods and materials can, for example, provide γδ T cells that can be used in allogeneic or autologous recipient subjects for the treatment of a variety of pathological conditions including, for example, viral infections, bacterial infections, fungal infections, protozoal infections and cancers.

Embodiments of the invention include expansion medias and methods for their use as well as functional CD16^{Hi} Vδ2 gamma delta T cells and populations of cells produced by the methods disclosed herein. Typically, these populations consist essentially of functional gamma delta T cells (e.g., do not include conventional αβ T cells). Specific embodiments of the invention include a cell culture media for expanding T lymphocytes that express CD16 in combination with Vδ2, the media comprising: a γδ T cell stimulator (e.g., bisphosphonate), a combination of cytokines, a combination of small molecules (e.g., Wnt activators, GSK-3β inhibitors). The medium can be a serum-containing or serum-free medium, or xeno-free medium. From the aspect of preventing contamination with heterogeneous animal-derived components, serum can be derived from the same animal as that of the stem cell(s). The serum-free medium refers to a medium with no unprocessed or unpurified serum and accordingly, can include medium with purified blood-derived components or animal tissue-derived components (such as growth factors).

Embodiments of the invention include methods of growing mammalian (e.g. human) cells, the methods comprising: obtaining a population of lymphocytes (e.g. from peripheral blood of one or more donors); identifying and/or selecting cell populations from one or more donors based on their Vδ2 T cell CD16 expression profile; and then expanding the identified, selected and/or purified T lymphocytes that express CD16 in combination with Vδ2. In this context, aspects of human γδ T Cell Subsets and mechanism of Vδ2 TCR stimulation are discussed in Lee et al., Cancers 2022, 14, 3005, the contents of which are incorporated by reference. In some embodiments of the invention, the population of lymphocytes is selected from one or more donors identified as having at least 35% CD16 positive cells within the population of Vδ2 T lymphocytes (termed herein “CD16^{Hi}”) obtained from the individual. In particular, as used herein, “CD16 high” means a cut-off of 35% or above CD16-positive cells of total Vδ2 (e.g., Vg9Vd2) T cells from primary donor PBMCs measured using flow cytometry. In other embodiments of the invention, the population of lymphocytes is selected from one or more individuals identified as having less than 35% CD16 positive cells within the population of Vδ2 T lymphocytes (termed herein “CD16^{Lo}”) obtained from the individual (e.g., as observed in a flow cytometry analysis).

In certain embodiments of the invention, the cells are expanded by 10, 100, 500, 1000, 2,000, 3000,4000, 5000, 6000, 7000, 8000, 9000, or 10,000 fold. In some embodiments, the expansion period lasts at least 3, 4,5 or 6 weeks. In some embodiments, the cells are restimulated one or more times in this culture. Typically in such methods, the identified, selected and/or purified T lymphocytes that express CD16 in combination with Vδ2 are expanded by disposing the T lymphocytes in a media comprising a γδ T cell stimulator (e.g., an antibody specific for the gamma delta receptor on the cells, a bisphosphonate such as zoledonic acid or the like), a combination of cytokines (e.g. IL-2, IL-15, IL-12, IL-18, IL-7, IL-21, TGF-β), and a Wnt activator (e.g. Wnt3a) and/or a GSK-3β inhibitor (e.g. TWS119). For example, in certain embodiments, the media comprises IL-7 in combination with IL-21. In some embodiments of the invention, the media comprises a bisphosphonate such as Zoledonic acid at a concentration from about 0.1 uM to about 100 uM (e.g., 5-10 uM). In some embodiments of the invention, the media comprises a phosphoantigen such as (E)-4-hydroxy-3-methyl-but-2-enyl pyrophosphate (HMBPP) at a concentration from about 1 nM to about 1 uM. In certain embodiments of the invention, the media comprises one or more cytokines at a concentration from about 1 ng/ml to about 500 ng/mL. In certain embodiments of the invention, the media comprises a Wnt activator (e.g., Wnt 3a) at a concentration from about 1 ng/ml to about 1 ug/mL. In certain embodiments of the invention, the media comprises a GSK-3β inhibitor (e.g., TWS119) at a concentration from about 0.1 uM to about 50 uM.

In some embodiments, CD16^{Hi} Vδ2 T cells are cultured and/or expanded in medium containing 1, 2, 3, 4, 5, 6, 7, or 8 of the following Wnt activators and/or GSK-3β inhibitors: Wnt3A, CHIR99021, AR-A014418, TWS119, LY2090314, 9-ING-41, lithium chloride (LiCl), or BIO (6-bromoindirubin-3-oxime, 6-Bromoindirubin-3′-oxime). In additional embodiments, CD16^{Hi} Vδ2 T cells are cultured and/or expanded in medium containing 1, 2, 3, or 4 of the following tyrosine kinase inhibitors: dasatinib, ibrutinib, acalabrutinib, or zanubrutinib.

In typical embodiments, CD16^{Hi} Vδ2 T cells are cultured and/or expanded in serum-free medium. In certain embodiments, the serum-free medium further comprises 1, 2, 3, 4, 5, 6, 7, 8, 9, 10, 11, 12, 13, 14, 15, or all 16 (or a range derivable therein) of the following externally added components:, interleukin 7 (IL-7), stromal derived factor 1α (SDF-1α), IL-2, IL-4, IL-6, IL-12, IL-15, IL-18, IL-21, IL-23, TNF-alpha, TGF-beta, interferon-gamma, interferon-lambda, TSLP, thymopentin, pleotrophin, or midkine. In additional embodiments, the serum-free medium comprises one or more vitamins. In some cases, the serum-free medium includes 1, 2, 3, 4, 5, 6, 7, 8, 9, 10, 11, or 12 of the following vitamins (or any range derivable therein): comprise biotin, DL alpha tocopherol acetate, DL alpha-tocopherol, vitamin A, vitamin C, choline chloride, calcium pantothenate, pantothenic acid, folic acid nicotinamide, pyridoxine, riboflavin, thiamine, inositol, vitamin B12, or a salt thereof. In certain embodiments, medium comprises or comprise at least biotin, DL alpha tocopherol acetate, DL alpha-tocopherol, vitamin A, vitamin C, or combinations or salts thereof. In additional embodiments, serum-free medium comprises one or more proteins. In some embodiments, serum-free medium comprises 1, 2, 3, 4, 5, 6 or more (or any range derivable therein) of the following proteins: albumin or bovine serum albumin (BSA), a fraction of BSA, catalase, insulin, transferrin, superoxide dismutase, or combinations thereof. In other embodiments, serum-free medium comprises 1, 2, 3, 4, 5, 6, 7, 8, 9, 10, or 11 of the following compounds: corticosterone, D-Galactose, ethanolamine, glutathione, L-carnitine, linoleic acid, linolenic acid, progesterone, putrescine, sodium selenite, or triodo-I-thyronine, or combinations thereof. In further embodiments, serum-free medium comprises a B-27® supplement, xeno-free B-27® supplement, GS21TM supplement, or combinations thereof. In additional embodiments, serum-free medium comprises or further comprises amino acids, monosaccharides, and/or inorganic ions. In some aspects, serum-free medium comprises 1, 2, 3, 4, 5, 6, 7, 8, 9, 10, 11, 12, or 13 of the following amino acids: arginine, cysteine, isoleucine, leucine, lysine, methionine, glutamine, phenylalanine, threonine, tryptophan, histidine, tyrosine, or valine, or combinations thereof. In other aspects, serum-free medium comprises 1, 2, 3, 4, 5, or 6 of the following inorganic ions: sodium, potassium, calcium, magnesium, nitrogen, or phosphorus, or combinations or salts thereof. In additional aspects, serum-free medium comprises 1, 2, 3, 4, 5, 6 or 7 of the following elements: molybdenum, vanadium, iron, zinc, selenium, copper, or manganese, or combinations thereof. In certain embodiments, the serum-free medium further comprises externally added ascorbic acid. In particular embodiments, methods involve adding ascorbic acid medium.

The medium in certain embodiments of the invention can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In typical embodiments of the invention, the expansion medium is a serum-free medium that is suitable for cell development. For example, the medium may comprise B-27® supplement, xeno-free B-27® supplement (available at world wide web at thermofisher. com/us/en/home/technical-resources/media-formulation.250.html), NS21 supplement (Chen et al., J Neurosci Methods, 2008 June 30; 171(2): 239-247, incorporated herein in its entirety), GS21^{TM} supplement (available at world wide web at amsbio. com/B-27.aspx), or a combination thereof at a concentration effective for producing T cells from the 3D cell aggregate.

As discussed in the Example below, in some embodiments, a CD16^{Hi} Vδ2 T cell of the invention is disposed in selected media conditions during growth and differentiation. Cells produced by the preparation methods may be frozen. The produced cells may be in a solution comprising dextrose, one or more electrolytes, albumin, dextran, and DMSO. The solution may be sterile, nonpyogenic, and isotonic. In some embodiments, the γδ T cell has previously been frozen and the previously frozen cell is stable at room temperature for at least one hour. In some embodiments, the CD16^{Hi} Vδ2 T cell has previously been frozen, and the previously frozen cell is stable at room temperature for at least 1, 2, 3, 4, 5, 6, 7, 8, 10, 15, 20, 24, 30, or 48 hours (or any derivable range therein). In certain embodiments, a CD16^{Hi} Vδ2 T cell or a population of CD16^{Hi} Vδ2 T cells in a solution comprises dextrose, one or more electrolytes, albumin, dextran, and/or DMSO.

As discussed below, in certain embodiments of the invention, PBMCs are cultured to generate memory-like Vδ2 T cells (The figures show cellular markers associated with memory-like Vδ2 T cells such as CD27 and CD45RA surface markers). Vδ2 T cells can be stimulated and expanded by supplementing into the cell culture Vδ2-Stimulatory Reagents, including but not limited to, TCR cognate antigens, phosphoantigens, small molecules, and/or antibodies. Examples of such reagents include isopentenyl pyrophosphate, zoledronate, pamidronate, risedronate, alendronate, ibandronate, tiludronate, etidronate, anti-γδTCR antibodies, non-specific TCR stimulatory reagents (anti-CD3/anti-CD28 antibodies or antibody-coated beads, Concanavalin A, PMA/Ionomycin, and artificial APCs), anti-CD16 antibodies, and others. Memory-like feature of the cultured Vδ2 T cells can be achieved by supplementing into the cell culture Memory-Promoting Reagents, including but not limited to, serum albumin, L-ascorbic acid, 2-mercaptoethanol, IL-2, IL-4, IL-7, IL-12,IL-15, IL-18, IL-21, IL-23, TNFα, SDF-1α, TGF-β, and Wnt activators or glycogen synthase kinase-3 (GSK-3) inhibitors such as Wnt3A, CHIR99021, AR-A014418, TWS119, LY2090314, 9-ING-41, lithium chloride (LiCl), BIO (6-bromoindirubin-3-oxime, 6-Bromoindirubin-3′-oxime). The Memory Medium may also include tyrosine kinase inhibitors such as dasatinib, ibrutinib, acalabrutinib, zanubrutinib, and others. The Memory Medium can be used to culture and expand all types of Vδ2 T cells, including CD16H Vδ2 T cells. In some embodiments, CD16^{Lo} Vδ2 T and/or mixture of CD16^{Lo} Vδ2 T and CD16^{Hi} Vδ2 T cells are cultured/expanded using the aforementioned medias and methods.

In embodiments involving multiple cells, a CD16^{Hi} Vδ2 T cell population may comprise, comprise at least, or comprise at most about 10², 10³, 10⁴, 10⁵, 10⁶, 10⁷, 10⁸, 10⁹, 10¹⁰, 10¹¹, 10¹², 10¹³, 10¹⁴, 10¹⁵ cells or more (or any range derivable therein), which are CD16^{Hi} Vδ2 T cells in some embodiments. In some cases, a cell population comprises at least about 10⁶-10¹² CD16^{Hi} Vδ2 T cells. It is contemplated that in some embodiments, that a population of cells with these numbers is produced from a single batch of cells and are not the result of pooling batches of cells separately produced.

Embodiments of the invention also include methods of treating patients with a CD16^{Hi} Vδ2 T cell or cell population as disclosed herein. Such embodiments of the invention include methods of treating a subject in need of gamma delta T cells (e.g., to fight a disease such as an autoimmune disease or a cancer or an infection such as COVID-19) which comprises administering to the subject a functional CD16^{Hi} Vδ2 T cell disclosed herein. In this way, such T cells may be used to treat patients having a wide variety of pathological conditions. In certain therapeutic methods of the invention, the patient has been diagnosed with a cancer. In some embodiments of the invention, the patient has a disease or condition involving inflammation, which, in some embodiments, excludes cancer. In certain embodiments, the patient has an autoimmune disease or condition. In particular aspects of the invention, the cells or cell population is allogeneic with respect to the patient. In certain embodiments, the patient does not exhibit signs of rejection or depletion of the cells or cell population. Some therapeutic methods further include administering to the patient an antibody or stimulatory molecule (e.g., alone or loaded onto APCs) that activates γδ T cells, and/or a compound that initiates a suicide gene product.

Treatment of a cancer patient with the CD16^{Hi} Vδ2 T cells may result in tumor cells of the cancer patient being killed after administering the CD16^{Hi} Vδ2 T cells or cell population to the patient. Treatment of an inflammatory disease or condition may result in reducing inflammation. In other embodiments, a patient with an autoimmune disease or condition may experience an improvement in symptoms of the disease or condition or may experience other therapeutic benefits from the CD16^{Hi} Vδ2 T cell populations disclosed herein. Combination treatments with CD16^{Hi} Vδ2 T cells and standard therapeutic regimens or another immunotherapy regimen(s) may be employed. As discussed below, in some embodiments, the CD16^{Hi} Vδ2 T cells can be engineered to modulate endogenous genes and/or express exogenous transgenes. Embodiments of the invention include compositions of matter comprising an CD16^{Hi} Vδ2 T cell or T cell population disclosed herein such as one comprising a gene expression profile characterized as: HLA-I-negative; HLA-II-negative; HLA-E-positive; expressing a suicide gene; and expressing one or more exogenous nucleic acids such as those encoding a T cell receptor gamma chain polypeptide and an exogenous T cell receptor delta chain polypeptide. Optionally, the CD16^{Hi} Vδ2 T cell further comprises an exogenous nucleic acid molecule encoding another polypeptide such as a T cell receptor alpha chain polypeptide and/or a T cell receptor beta chain polypeptide and/or an iNKT receptor polypeptide; and/or a cytokine; and/or comprises suppressed endogenous TCRs.

In certain embodiments, a CD16^{Hi} Vδ2 T cell is lacking or has reduced surface expression of at least one HLA-I or HLA-II molecule. In some embodiments, the lack of surface expression of HLA-I and/or HLA-II molecules is achieved by disrupting the genes encoding individual HLA-I/II molecules, or by disrupting the gene encoding B2M (beta 2 microglobulin) that is a common component of all HLA-I complex molecules, or by disrupting the genes encoding CIITA (the class II major histocompatibility complex transactivator) that is a critical transcription factor controlling the expression of all HLA-II genes. In specific embodiments, the cell lacks the surface expression of one or more HLA-I and/or HLA-II molecules, or expresses reduced levels of such molecules by (or by at least) 50, 60, 70, 80, 90, 100% (or any range derivable therein). In some embodiments, the HLA-I or HLA-II are not expressed in the γδ T cell because the cell was manipulated by gene editing. In some embodiments, the gene editing involved is CRISPR-Cas9. Instead of Cas9, CasX or CasY may be involved. Zinc finger nuclease (ZFN) and TALEN are other gene editing technologies, as well as Cpfl, all of which may be employed. In other embodiments, the γδ T cell comprises one or more different siRNA or miRNA molecules targeted to reduce expression of HLA-I/II molecules, B2M, and/or CIITA.

In a particular embodiment, there is a CD16^{Hi} Vδ2 T cell that expresses an exogenous nucleic acid such as one encoding a suicide gene product. Methods in the art for suicide gene usage may be employed, such as in U.S. Pat. No. 8,628,767, U.S. Patent Application Publication 20140369979, U.S. 20140242033, and U.S. 20040014191, all of which are incorporated by reference in their entirety. In further embodiments, a TK gene is a viral TK gene, i.e. a TK gene from a virus. In particular embodiments, the TK gene is a herpes simplex virus TK gene. In some embodiments, the suicide gene product is activated by a substrate. Thymidine kinase is a suicide gene product that is activated by ganciclovir, penciclovir, or a derivative thereof. In certain embodiments, the substrate activating the suicide gene product is labeled in order to be detected. In some instances, the substrate that may be labeled for imaging. In some embodiments, the suicide gene product may be encoded by the same or a different nucleic acid molecule encoding one or both of TCR-gamma or TCR-delta. In certain embodiments, the suicide gene is sr39TK or inducible caspase 9. In alternative embodiments, the cell does not express an exogenous suicide gene.

In specific embodiments, there is an T cell population comprising: clonal CD16^{Hi} Vδ2 T cells comprising one or more exogenous nucleic acids encoding molecules such as an γδ T-cell receptor and a thymidine kinase suicide gene product, wherein the clonal γδ T cells have been engineered not to express functional beta-2-microglobulin (B2M), and/or class II, major histocompatibility complex, or transactivator (CIITA) and wherein the cell population is at least about 10⁶-10¹² total cells and comprises at least about 10²-10⁶ γδ T cells. In certain instances, the cells are frozen in a solution.

In some embodiments, a CD16^{Hi} Vδ2 T cell of the invention comprises a recombinant vector or a nucleic acid sequence from a recombinant vector that was introduced into the γδ cells. In certain embodiments the recombinant vector is or was a viral vector. In further embodiments, the viral vector is or was a lentivirus, a retrovirus, an adeno-associated virus (AAV), a herpesvirus, or adenovirus. It is understood that the nucleic acid of certain viral vectors integrate into the host genome sequence.

In typical embodiments, an exogenous nucleic acid may be transduced into the CD16^{Hi} Vδ2 T cells such as one that comprises a nucleotide sequence encoding a γ-TCR and/or a δ-TCR. In certain embodiments, one nucleic acid encodes both the gamma and delta chains of the TCR. In some embodiments, a further nucleic acid may comprise a nucleic acid sequence encoding an α-TCR and/or a β-TCR polypeptide, and/or one or more iNKT TCR polypeptides. In additional embodiments, a nucleic acid further comprises a nucleic acid sequence encoding a suicide gene product. In some embodiments, a nucleic acid molecule that is introduced into a selected cell encodes the TCR, and the suicide gene product. In other embodiments, a method also involves introducing into the selected cells a nucleic acid encoding a suicide gene product, in which case a different nucleic acid molecule encodes the suicide gene product than a nucleic acid encoding at least one of the TCR genes.

As discussed above, in some embodiments the CD16^{Hi} Vδ2 T cells do not express the HLA-I and/or HLA-II molecules on the cell surface, which may be achieved by disrupting the expression of genes encoding beta-2-microglobulin (B2M), transactivator (CIITA), or HLA-I and HLA-II molecules. In certain embodiments, methods involve eliminating surface expression of one or more HLA-I/II molecules in the isolated human cells. In particular embodiments, eliminating expression may be accomplished through gene editing of the cell's genomic DNA. Some methods include introducing CRISPR and one or more guide RNAs (gRNAs) corresponding to B2M or CIITA into the cells. In particular embodiments, CRISPR or the one or more gRNAs are transfected into the cell by electroporation or lipid-mediated transfection. Consequently, methods may involve introducing CRISPR and one or more gRNAs into a cell by transfecting the cell with nucleic acid(s) encoding CRISPR and the one or more gRNAs. A different gene editing technology may be employed in some embodiments. Similarly, in some embodiments, one or more nucleic acids encoding the TCR receptor are introduced into the cell. This can be done by transfecting or infecting the cell with a recombinant vector, which may or may not be a viral vector as discussed herein. The exogenous nucleic acid may incorporate into the cell's genome in some embodiments.

In some embodiments, methods include introducing one or more additional nucleic acids into the cell population, which may or may not have been previously frozen and thawed. This use provides one of the advantages of creating an off-the-shelf CD16^{Hi} Vδ2 T cell. In particular embodiments, the one or more additional nucleic acids encode one or more therapeutic gene products. Examples of therapeutic gene products include at least the following: 1. Antigen recognition molecules, e.g. CAR (chimeric antigen receptor) and/or TCR (T cell receptor); 2. Co-stimulatory molecules, e.g. CD28,4-1BB, 4-1BBL, CD40, CD40L, ICOS; and/or 3. Cytokines, e.g. IL-1α, IL-1β, IL-2, IL-4, IL-6, IL-7, IL-9, IL-15, IL-12, IL-17, IL-21, IL-23, IFN-γ, TNF-α, TGF-β, G-22 CSF, GM-CSF; 4. Transcription factors, e.g. T-bet, GATA-3, RORγt, FOXP3, and Bcl-6. Therapeutic antibodies are included, as are chimeric antigen receptors, single chain antibodies, monobodies, humanized, antibodies, bi-specific antibodies, single chain FV antibodies or combinations thereof.

Aspects of the disclosure relate to a human cell comprising: i) an exogenous expression or activity inhibitor of; or ii) a genomic mutation of: one or more of β₂ microglobin (B2M), CIITA, TRAC, TRBC1, or TRBC2. In some embodiments, the cell comprises a genomic mutation. In some embodiments, the genomic mutation comprises a mutation of one or more endogenous genes in the cell's genome, wherein the one or more endogenous genes comprise the B2M, CIITA, TRAC, TRBC1, or TRBC2 gene. In some embodiments, the mutation comprises a loss of function mutation. In some embodiments, the inhibitor is an expression inhibitor. In some embodiments, the inhibitor comprises an inhibitory nucleic acid. In some embodiments, the inhibitory nucleic acid comprises one or more of a siRNA, shRNA, miRNA, or an antisense molecule. In some embodiments, the cells comprise an activity inhibitor. In some embodiments, following modification the cell is deficient in any detectable expression of one or more of B2M, CIITA, TRAC, TRBC1, or TRBC2 proteins. In some embodiments, the cell comprises an inhibitor or genomic mutation of B2M. In some embodiments, the cell comprises an inhibitor or genomic mutation of CIITA. In some embodiments, the cell comprises an inhibitor or genomic mutation of TRAC. In some embodiments, the cell comprises an inhibitor or genomic mutation of TRBC1. In some embodiments, the cell comprises an inhibitor or genomic mutation of TRBC 2. In some embodiments, at least 90% of the genomic DNA encoding B2M, CIITA, TRAC, TRBC1, and/or TRBC2 is deleted. In some embodiments, at least or at most 5, 10, 20, 30, 40, 50, 60, 70, 80, 90, 95, 99, or 100% (or any range derivable therein) of the genomic DNA encoding B2M, CIITA, TRAC, TRBC1, and/or TRBC2 is deleted. In other embodiments, a deletion, insertion, and/or substitution is made in the genomic DNA. In some embodiments, the cell is a progeny of the human stem or progenitor cell.

In cases wherein the CD16^{Hi} Vδ2 T cells comprise one or more suicide genes for subsequent depletion upon need, the suicide gene may be of any suitable kind. The γδ T cells of the disclosure may express a suicide gene product that may be enzyme-based, for example. Examples of suicide gene products include herpes simplex virus thymidine kinase (HSV-TK), purine nucleoside phosphorylase (PNP), cytosine deaminase (CD), carboxypetidase G2, cytochrome P450, linamarase, beta-lactamase, nitroreductase (NTR), carboxypeptidase A, or inducible caspase 9. Thus, in specific cases, the suicide gene may encode thymidine kinase (TK). In specific cases, the TK gene is a viral TK gene, such as a herpes simplex virus TK gene. In particular embodiments, the suicide gene product is activated by a substrate, such as ganciclovir, penciclovir, or a derivative thereof.

In some embodiments, the CD16^{Hi} Vδ2 T cells are able to be imaged or otherwise detected. In particular cases, the cells comprise an exogenous nucleic acid encoding a polypeptide that has a substrate that may be labeled for imaging, and the imaging may be fluorescent, radioactive, colorimetric, and so forth. In specific cases, the cells are detected by positron emission tomography. The cells in at least some cases express sr39TK gene that is a positron emission tomography (PET) reporter/thymidine kinase gene that allows for tracking of these genetically modified cells with PET imaging and elimination of these cells through the sr39TK suicide gene function.

Encompassed by the disclosure are populations of CD16^{Hi} Vδ2 T cells. In particular aspects, CD16+Vδ2 T clonal cells comprise exogenous nucleic acids such as ones encoding an γδ T-cell receptor and lack surface expression of one or more HLA-I or HLA-II molecules. The γδ T cells may comprise an exogenous nucleic acid encoding a suicide gene, including an enzyme-based suicide gene such as thymidine kinase (TK). The TK gene may be a viral TK gene, such as a herpes simplex virus TK gene. In the cells of the population the suicide gene may be activated by a substrate, such as ganciclovir, penciclovir, or a derivative thereof, for example. The cells may comprise an exogenous nucleic acid encoding a polypeptide that has a substrate that may be labeled for imaging, and in some cases a suicide gene product is the polypeptide that has a substrate that may be labeled for imaging. In specific aspects, the suicide gene is sr39TK. In particular cases for the γδ T cell population, the γδ T cells comprise nucleic acid sequences from a recombinant vector that was introduced into the cells, such as a viral vector (including at least a lentivirus, a retrovirus, an adeno-associated virus (AAV), a herpesvirus, or adenovirus).

In certain embodiments, the cells of the CD16^{Hi} Vδ2 T cell population may or may not have been exposed to, or are exposed to, one or more certain conditions. In certain cases, for example, the cells of the population not exposed or were not exposed to media that comprises animal serum. The cells of the population may or may not be frozen. In some cases, the cells of the population are in a solution comprising dextrose, one or more electrolytes, albumin, dextran, and/or DMSO. The solution may comprise dextrose, one or more electrolytes, albumin, dextran, and DMSO. The cells may be in a solution that is sterile, nonpyogenic, and isotonic. In specific cases the yo T cells have been activated, such as activated with ZOL. In specific aspects, the cell population comprises at least about 10²-10⁶ clonal cells. The cell population may comprise at least about 10⁶-10¹² total cells, in some cases.

In particular embodiments there is a CD16^{Hi} Vδ2 T cell population comprising: clonal CD16^{Hi} Vδ2 T cells comprising one or more exogenous nucleic acids encoding an γδ T-cell receptor and a thymidine kinase suicide, wherein the clonal γδ T cells have been engineered not to express functional beta-2-microglobulin (B2M), major histocompatibility complex class II transactivator (CIITA), and/or HLA-I and HLA-II molecules and wherein the cell population is at least about 10⁶-10¹² total cells and comprises at least about 10²-10⁶ clonal cells. In some cases, the cells are frozen in a solution.

As noted above, genetic modification may also be introduced to certain components to generate antigen-specific T cells, and to model positive and negative selection. Examples of these modifications include transduction of HSCs with a lentiviral vector encoding an antigen-specific T cell receptor (TCR) or chimeric antigen receptor (CAR) for the generation of antigen-specific, allelically excluded naïve T cells; transduction of HSCs with gene/s to direct lineage commitment to specialized lymphoid cells.

### EXAMPLES

### Example 1: Use of Allogeneic Vδ2 T Cells for Ovarian Cancer Therapy Through CD16 Biomarker Selection and CAR/IL-15 Engineering

The recent success of chimeric antigen receptor (CAR)-T cell therapies in treating hematological malignancies highlights the transformative potential of genetically engineered cell therapies¹⁻³. CARs are fusion proteins linking a targeting moiety, typically the single chain variable fragment of an antibody, to T cell stimulatory domains, allowing CAR-engineered cells to both target and kill cancer cells⁴. Conventional αβ T cells are the chassis for six FDA approved CAR-engineered cell products, targeting CD19 for the treatment of B cell malignancies and B-cell maturation antigen (BCMA) for multiple myeloma. CAR-T therapies have also been actively tested in solid tumor settings with limited therapeutic benefit⁵, although GD2− and Claudin18.2-targeting CAR-T cells display encouraging results in small patient cohorts^{6,7}. Solid tumors present several challenges to CAR-T cells, including barriers to infiltration, antigen heterogeneity, and immunosuppressive tumor microenvironments (TME)⁸. In addition to solid tumor efficacy concerns, CAR-T cells can cause severe adverse events, such as cytokine release syndrome (CRS), and the autologous nature of CAR-T cell therapies limits their accessibility⁹. Complex manufacturing and patient-derived starting material further result in exorbitant costs, time constraints, and final product variability.

Allogeneic cell therapies hold promise in addressing limitations of the present-day CAR-T cell paradigm¹⁰. Extensive research with αβ T cells and their clinical validation in autologous products has spurred active investigation into developing allogeneic αβ CAR-T cells. To circumvent the risk of graft-versus-host disease (GvHD) due to the recognition of mismatched MHC molecules by endogenous af TCRs, gene editing is often required. CAR-engineering antigen-specific αβ T cells, such as CMV-specific T cells, is another approach to avoid GvHD and create off-the-shelf αβ CAR-T cells. Other alternative strategies center on innate and innate-like immune cell populations that are inherently safer for allogeneic transfer, such as macrophages, natural killer (NK) cells, invariant natural killer T (iNKT) cells, and γδ T cells^{11,12}.

T cells expressing T cell receptors (TCRs) composed of γ and δ chains, γδ T cells, only make up 1-10% of circulating T cells but have conserved antimicrobial and antitumor functions¹³. γδT cells display characteristics of both innate and adaptive immune systems, mediating cytotoxicity through both TCR and NK receptor signaling and expressing an array of context-dependent immunomodulatory cytokines^{14,15}. The cancer-fighting potential of γδ T cells has been spotlighted by Gentles et al., in which tumor-infiltrating γδ T cells were the strongest favorable prognosis factor of all immune cell subsets in several hematological and solid cancers¹⁶. As non-peptide-MHC dependent responders, γδ T cells are not expected to cause GvHD and have proven to be safe in clinical allogeneic settings¹⁷.

Upwards of 90% of peripheral γδ T cells possess a Vγ9Vδ2 TCR, which senses elevated phosphorylated nonpeptide metabolites, or phosphoantigens (pAg)¹⁸. Specifically, dysregulation of the mevalonate metabolism causes an accumulation of intracellular pAgs, which results in conformational changes in B7-related membrane protein butyrophilin (BTN) 3A1 that allows BTN3A1 interaction with BTN2A1, and this complex is recognized by the Vγ9Vδ2 TCR^{19,20}. Dysregulated cellular energetics is an emerging hallmark of cancer²¹ and thus, the pAg/BTN pathway empowers TCR-dependent killing of a wide variety of liquid and solid tumors by Vγ9Vδ2 T cells²². Stimulation of the cells can also be accomplished using bisphosphonates, which are a class of drugs that prevent or slow down bone loss. Examples of such drugs include Zoledronate (ZOL) and Pamidronate (PAM). These small molecule compounds inhibit farnesyl pyrophosphate synthase in the mevalonate metabolic pathway. As a result, pAgs accumulate in the treated cells, leading to activation of the Vγ9Vδ2 TCR.

Despite the favorable attributes that encourage the development of Vγ9Vδ2 (Vδ2) T cells as cell carriers for off-the-shelf CAR therapies, literature investigating CAR-Vδ2 T cells is limited, especially compared to those investigating conventional αβ T cells. Furthermore, engineering therapeutic cells with transgenic IL-15, which has proven to enhance the preclinical efficacy of CAR-engineered NK²³, iNK²⁴, NKT²⁵, and Vδ1 T²⁶ cells, has not been reported for CAR-Vδ2 T cells.

CD16 (FcγRIII) is well-established as an IgG receptor that mediates antibody-dependent cell-mediated cytotoxicity (ADCC). Previous studies have explored the presence and functionality of CD16 on Vδ2 T cells²⁷⁻³⁰. In this work, we report a simple method for generating Vδ2 T cells with enhanced antitumor activity by screening donors for CD16 expression on Vδ2 T cells. Vδ2 T cells derived from CD16^{Hi} donors exhibit phenotypic traits that are advantageous for cancer treatment, including increased expression of effector molecules and ADCC activity. Additionally, we employ mesothelin-targeted CAR and IL-15 engineering to further enhance the antitumor potential of CD16^{Hi} Vδ2 T cells. Our results highlight the feasibility, therapeutic potential, and high safety profile of engineered CD16^{Hi} Vδ2 T cells in the context of cancer treatment.”

### RESULTS

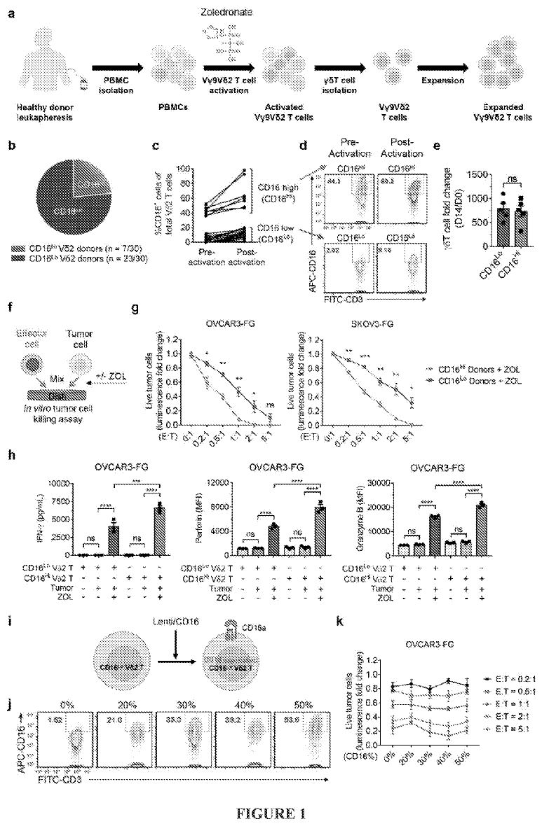

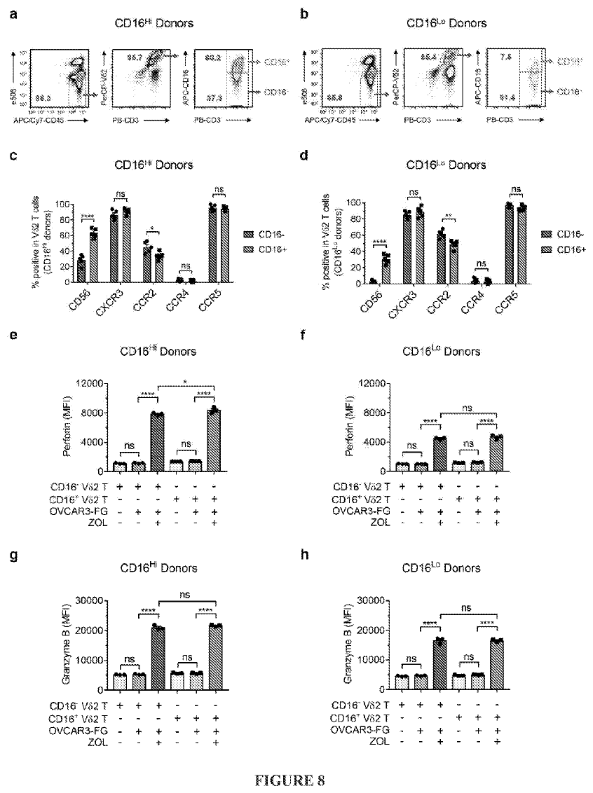

CD16 serves as a biomarker to screen PBMC donors for high performance Vδ2 T cells. The expansion of Vγ9Vδ2 (Vδ2) T cells from a large cohort of peripheral blood mononuclear cell (PBMC) donors for the development of γδ T cell-based cancer therapies revealed notable differences in CD16 expression (FIG. 1a-d; FIG. 8a, b). The initial CD16 expression on PBMC-derived Vδ2 T cells (before stimulation) ranged from nearly lack of CD16 expression to over 35%. CD16 high (CD16^{Hi}) Vδ2 T cells and CD16 low (CD16^{Lo}) Vδ2 T cells were defined as ≥35% CD16 expression and ≤20% CD16 expression, respectively. A total of 30 healthy donors were screened for this experiment, in which 7 (23.3%) were classified as CD16^{Hi} donors (FIG. 1b). Notably, CD16 expression on CD16^{Hi} Vδ2 T cells was not only maintained but also increased upon Vδ2 T cell activation with ZOL and expansion for 14 days (FIG. 1c, d). Using the ZOL and IL-2 expansion method, we produced comparable expansion of Vδ2 T cells irrespective of CD16 expression (FIG. 1e).

Past studies have shown that Vδ2 T cells exert potent cytotoxicity against various types of tumors. We compared the killing of human ovarian tumor cells by Vδ2 T cells expanded from CD16^{Hi} and CD16^{Lo} donors. High-grade serous ovarian cancer cell lines, OVCAR3 and SKOV3, were engineered with firefly luciferase and green fluorescence protein dual reporters (FG) and cocultured with various ratios of effector cells (effector to tumor, E:T ratio) in the presence or absence of ZOL (FIG. 1f, g). Twenty-four hours post co-culture, tumor cell killing was measured by bioluminescence; three CD16^{Hi} and three CD16^{Lo} Vδ2 T cell donors were used. In the presence of ZOL, CD16^{Hi} Vδ2 T cells displayed significantly enhanced cytotoxicity at almost all the E:T ratios tested, for both cancer cell lines. The improved cytotoxicity of Vδ2 T cells expanded from CD16^{Hi} donors correlated with increased IFN-γ secretion, as measured by ELISA, and perforin and granzyme B production, as measured by intracellular staining, following 24 hours (h) co-culture of cancer cells and effector cells at a 1:1 E:T ratio in the presence or absence of ZOL (FIG. 1h). Depending on the cancer cell type and assay used, Vδ2 T cells are capable of tumor killing as well as cytokine, perforin, and granzyme B production in the absence of ZOL; in some cases, ZOL or other preconditioning is used to exert effective cancer killing^{31,32}. However, for the ovarian cancer cells we tested, Vδ2 T cells exhibited a dependency on ZOL for cytotoxicity and effector molecule production during in vitro cocultures (FIG. 1h). Although differences in cytotoxicity potential were observed, the expression of chemokine receptors CXCR3, CCR4, and CCR5 was comparable within donors and between donors, whereas CD56 was upregulated on CD16⁺ cells within donors and expressed at higher overall levels on Vδ2 T cells from CD16^{Hi} donors. CCR2 was upregulated on CD16⁻ cells within donors and expressed at overall higher levels on Vδ2 T cells from CD16^{Lo} donors (FIG. 8c, d). Importantly, the expression of granzyme B and perforin was similar between CD16⁺ and CD16⁻ Vδ2 T cells within a donor, and both types of cells displayed higher expression levels in Vδ2 T cells expanded from CD16^{Hi} donors than those expanded from CD16^{Lo} donors (FIG. 8e-h). These results indicate that Vδ2 T cells from CD16^{Hi} donors may be a favorable cell type for developing Vδ2 T cell-based cancer therapies.

We then created a lentiviral vector encoding CD16a³³ (Lenti/CD16) for engineering CD16^{Lo} Vδ2 T cells to express transgenic CD16a (FIG. 1i-j). Importantly, titration of CD16 expression did not affect cytotoxicity during in vitro cocultures with OVCAR3-FG cells (FIG. 1k). This provides evidence that CD16 could be used as a biomarker to select for donors with highly potent Vδ2 T cells rather than functioning as an active receptor that enhances tumor killing, and that genetic introduction of CD16 to CD16^{Lo} Vδ2 T cells may not recapitulate the heightened activity of Vδ2 T cells expanded from the CD16^{Hi} donors.

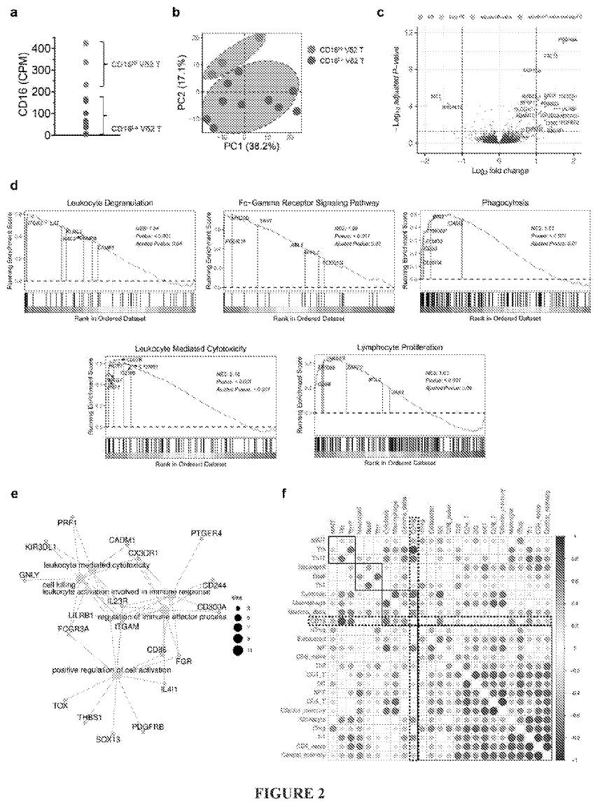

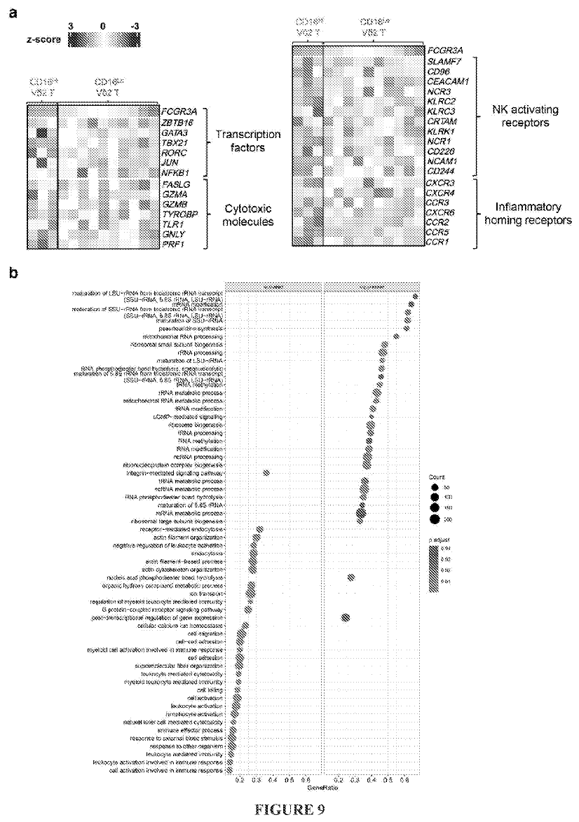

CD16^{Hi} Vδ2 T cells display enhanced cytotoxic gene signatures. To further assess the differences between Vδ2 T cells expanded from different donors, we performed bulk RNA-Seq on Vδ2 T cells expanded from 13 PBMC donors, 3 of which were classified as CD16^{Hi} based on our flow cytometry criteria. CD16 mRNA expression was assessed (FIG. 2a), and the samples were grouped into CD16^{Hi} and CD16^{Lo} Vδ2 T cells using k-means clustering algorithm (k=2). Three Vδ2 T cell samples were identified as CD16^{Hi} and the rest were CD16^{Lo}, confirming our flow cytometry results. Principal-component analysis (PCA) showed the clustering of CD16^{Hi} T cells, potentially indicating that there is transcriptomic variance between Vδ2 T cells correlating with CD16 expression (FIG. 2b). Differential expression analysis based on CD16 expression revealed an upregulation of genes associated with effector functions, such as GNLY, CD86, and CX3CR1, in high CD16 expressors (FIG. 2c). Various genes related to antitumor effector functions, such as genes encoding transcription factors, activation/homing markers, and cytotoxic molecules, were also assessed (FIG. 9a). CD16 expression positively correlated with the expression of granzymes (GZMA, GZMB), perforin (PRF1), and natural killer receptors (NCR1). Interestingly, the CD16^{Hi} group exhibited a downregulation of the transcription factor-encoding gene RORC, which is associated with a Th17-like phenotype. Th17-like Vδ2 T cells have been shown to promote cancer progression in several syngeneic cancer models and there is evidence for their detrimental effects in human malignancies³⁴⁻³⁶.

Furthermore, gene set enrichment analysis (GSEA)³⁷, were performed to characterize the biological pathway signatures associated with CD16 expression (FIG. 2d and FIG. 9b). High CD16 expression enriched for signatures associated with immune effector functions and activation, such as cytotoxicity, degranulation, Fc gamma receptor signaling, and phagocytosis. There was also an enrichment for proliferation-related genes in the CD16^{Hi} samples (FIG. 2d), although a difference in expansion was not observed during Vδ2 T cell in vitro production (FIG. 1e). A gene ontology (GO) over-representation Cnet plot³⁸ displaying the linkages of differentially expressed genes and biological processes confirmed the increase in immune cell activation signatures in CD16^{Hi} Vδ2 T cells (FIG. 2e).

We also assessed the relationship between CD16 expression in Vδ2 T cell samples and the gene signatures of 24 immune cell types defined in ImmuCellAI³⁹ (FIG. 2f). Enrichment scores of the immune cell types for each sample were calculated using single-sample GSEA (ssGSEA) implemented in the Gene Set Variation Analysis (GSVA) package⁴⁰. The resulting correlations between enrichment scores and the samples' CD16 expression level show that CD16 expression enriched for cytotoxic, NK, and macrophage signatures as well as an absence of Tfh and Th17 signatures. CD16 expression also corresponded to a Th1 signature, which is supported by the heightened secretion of IFN-γ by CD16^{Hi} Vδ2 T cells. In summary, the bulk RNA-Seq results align with the potent in vitro activity of CD16^{Hi} Vδ2 T cells and provide future directions for cell product characterization, especially with regard to IL-17 production and expansion potential.

Development of CAR- and IL-15-engineered CD16^{Hi} Vδ2 T cells targeting mesothelin for the treatment of ovarian cancer. Mesothelin (MSLN) is a cell-surface glycoprotein with limited expression on normal tissue but is overexpressed in many solid cancers, including ovarian, lung, and pancreatic carcinomas, making MSLN a promising target for cancer therapies, including CAR-engineered cell therapies^{41,42}. Due to the heterogenous expression of MSLN in ovarian cancer, we hypothesized that MSLN-targeting CAR (MCAR)-engineered Vδ2 T cells may exhibit superior antitumor activity due to the potential of multiple targeting capability. We also implemented cell engineering to produce IL-15 (termed MCAR15), as IL-15 signaling has been shown to enhance the persistence of innate/innate-like immune cells, and, to the best of our knowledge, has not yet been explored to modulate CAR Vδ2 T cells.

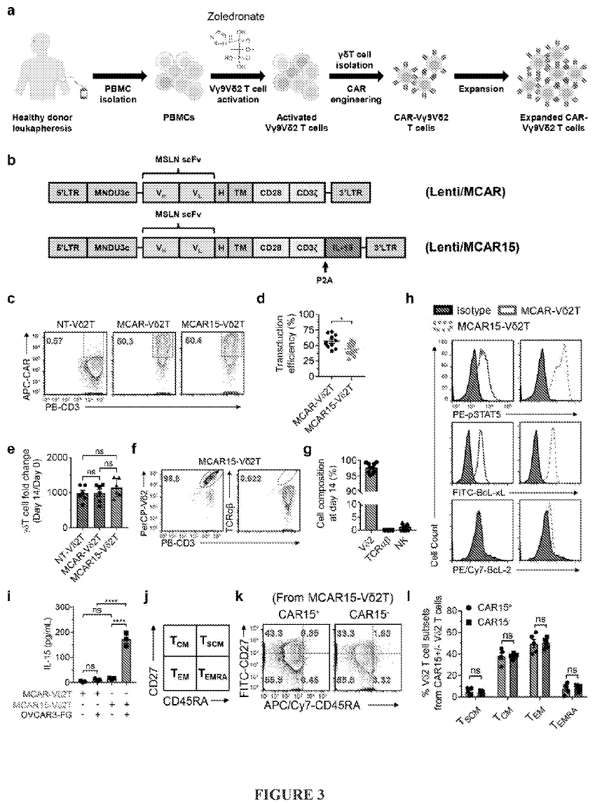

Following the schematic in FIG. 3a, we produced MCAR and MCAR15-engineered CD16^{Hi} Vδ2 T cells (MCAR-Vδ2T and MCAR15-Vδ2T) and included non-engineered CD16^{Hi} Vδ2 T cells as the control (NT-Vδ2T). MCAR and MCAR15 constructs resulted in similar CAR expression, Vδ2 T cell expansion, and Vδ2 T cell purity, with greater than 98% purity routinely achieved (FIG. 3b-g). MCAR15-Vδ2T cells expressed higher levels of persistence-associated proteins, pSTAT5, BcL-XL, and BcL-2 as measured by intracellular staining and flow cytometry (FIG. 3h). Marked IL-15 secretion was seen in the activated MCAR15 group (FIG. 3i) upon coculture with OVCAR3-FG cells, which is consistent with previously published data that demonstrate increased IL-15 production by CAR/IL-15 engineered αβ T⁴³, Vδ1 T²⁶, and NK cells²³ following antigen stimulation. This may be due to the heightened metabolic activity and protein translation that occur during cell activation as well as the short half-life of IL-15. Although various definitions of Vδ2 T cell memory status do exist in literature^{44,45}, our analysis based on CD27 and CD45RA expression showed that both CAR15⁺ and CAR15⁻ populations from the MCAR15-Vδ2T cell group were mostly central memory (~40%) and effector memory (~50%) phenotypes (FIG. 3j-l).

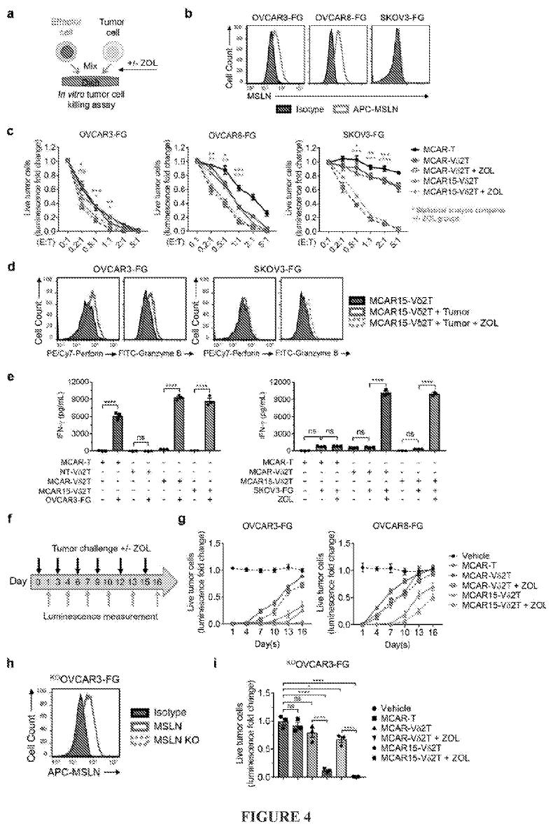

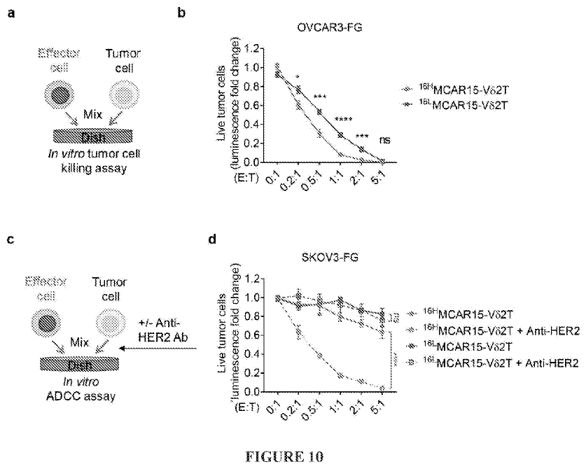

MCAR15-Vδ2T cells demonstrate robust in vitro antitumor activity against multiple ovarian cancer cell lines. We conducted in vitro cytotoxicity and cytokine production assays to evaluate the effector functions of CAR-engineered CD16^{Hi} Vδ2 T cells (FIG. 4). A third ovarian cancer cell line expressing FG dual reporter, OVCAR8-FG, was also included for several assays. The MSLN expression on OVCAR3-FG, OVCAR8-FG, and SKOV3-FG was assessed (FIG. 4b), revealing variable MSLN expression in the three ovarian cancer models. The conventional αβ CAR T cells targeting MSLN (MCAR-T) cells were included as a control, and Vδ2 T cell groups were added with and without ZOL unless otherwise specified. Following 24 h cocultures, all the effector cell groups exhibited efficient killing of OVCAR3-FG cancer cells (FIG. 4c). However, against OVCAR8-FG cells, Vδ2 T cell groups displayed heightened cytotoxicity compared to MCAR-T cells, and only in the presence of ZOL were Vδ2 T cells able to kill CAR-antigen negative SKOV3-FG cells (FIG. 4c). No differences were observed between the two engineered Vδ2 T cells groups (with and without IL-15). In parallel, we assessed the intracellular expression of granzyme B and perforin by MCAR15-Vδ2T cells co-cultured with OVCAR3-FG and SKOV3-FG cells in the presence or absence of ZOL (FIG. 4d). We observed an upregulation of effector molecule production in the presence of OVCAR3-FG cells with and without ZOL, whereas ZOL was required to increase effector molecule production in cocultures with SKOV3-FG cells. We also compared the in vitro cytotoxicity of CAR and IL-15 engineered CD16^{Hi} and CD16^{Lo} Vδ2 T cells and witnessed significantly improved killing by the CD16^{Hi} group (FIG. 10a, b).

Twenty-four hour cocultues were also used to monitor the IFN-γ production (FIG. 4e). When cultured with OVCAR3-FG, CAR-engineered T and Vδ2 T cells secreted ample IFN-γ, whereas non-engineered control Vδ2 T cells did not, highlighting the benefit of CAR-engineering. In corroboration with the cytotoxicity findings, IFN-γ was only produced in response to SKOV3-FG by CAR-Vδ2 T cells in the presence of ZOL. The cytotoxicity and IFN-γ results illustrate the tumor cell multi-targeting potential by CAR-Vδ2 T cells through both CAR and TCR recognition.

We performed in vitro repeated tumor challenge assays to investigate the long-term functionality of CAR15- and CAR-Vδ2 T cells (FIG. 4f, g). On days 0, 3, 6, 9, 12, and 15, effector cells were challenged with new tumor cells with and without ZOL in 96-well plates. Twenty-four hours following cancer cell addition, one of the 96-well plates was used for bioluminescence measurements to determine cancer killing. IL-15 secretion clearly improved cancer killing ability of MCAR15 Vδ2 T cell groups in repeated tumor challenges against OVCAR3-FG and OVCAR8-FG cells.

To further illustrate CAR-antigen dependent and independent antitumor activity, we generated MSLN-negative OVCAR3-FG (KOOVCAR3-FG) cells using CRISPR-Cas9 editing (FIG. 4h). ^{KO}OVCAR3-FG cells were cultured with CAR-engineered effector cells in repeated tumor challenges assays following the schematic shown in FIG. 4f. MSLN knockout resulted in reduced killing by CAR-Vδ2 T cells in the absence of ZOL compared to with ZOL after the repeated tumor challenge (FIG. 4i), whereas no difference was seen in cytotoxicity towards the parental OVCAR 3-FG cell line after 24 h cocultures (FIG. 4c). This indicates that in the absence of ZOL, the killing of parental OVCAR3-FG is driven by CAR-mediated killing. By day 7, after the 3^{rd} tumor challenge, KOOVCAR3-FG cell outgrowth occurred for all effector cell groups except for the MCAR- and MCAR15-Vδ2T cell cultures supplemented with ZOL. The MSLN knockout studies show that CAR-antigen presentation can enhance the killing functionality of CAR-Vδ2 T cells and CAR-antigen escape can potentially be overcome by the Vδ2 TCR-mediated killing mechanism.

Engineered CD16^{Hi} Vδ2 T cells can target ovarian cancer cells through ADCC. For several anticancer monoclonal antibodies (mAb) such as cetuximab and trastuzumab, Fc-mediated immune effector function, ADCC, is a major mode of action to deplete tumor cells^{46,47}. T and NK cell-mediated ADCC is predominantly attributed to the CD16a (FcγRIIIa) transmembrane receptor, which is expressed by many effector cells of the immune system, whereas CD16b (FcγRIIIb), a GPI-anchored protein, is exclusively expressed by neutrophils⁴⁸. Although CD32 (FcγRI) and CD64 (FcγRII) are expressed at low levels on Vδ2 T cells and may contribute to ADCC, they are primarily implemented in myeloid-mediated ADCC^{49,50}. We thus focus our Vδ2 T cell studies on the canonical lymphocyte CD16a receptor, which we refer to as CD16.

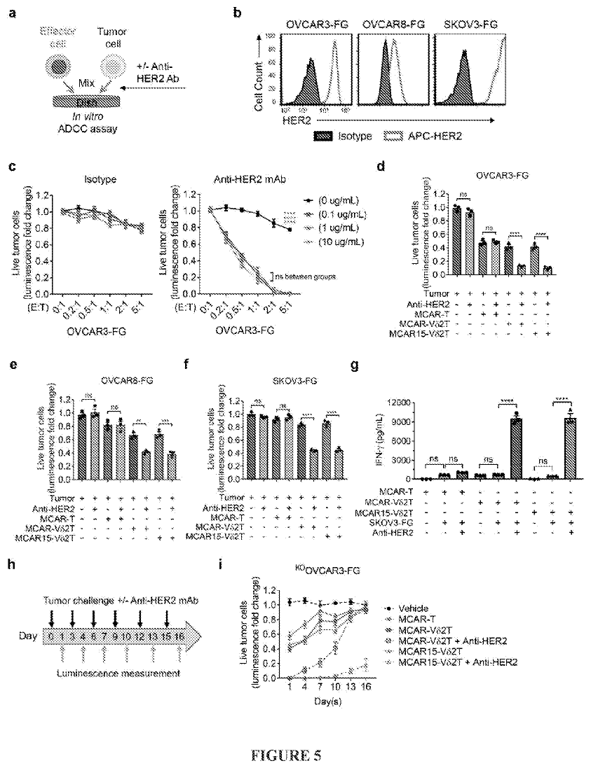

To test the ADCC capacity of unmodified and engineered CD16^{Hi} Vδ2 T cells, we performed in vitro tumor coculture assays with a preclinical anti-HER2 mAb analog to trastuzumab (FIG. 5a). The expression of HER2 on OVCAR3-FG, OVCAR8-FG, and SKOV3-FG was assessed by flow cytometry (FIG. 5b). For unmodified CD16^{Hi} Vδ2 T cells, against OVCAR3-FG cells, isotype control had minimal impact on Vδ2 T cell cytotoxicity, whereas significant enhancement of tumor killing was seen with concentrations of anti-HER2 mAb as low as 0.1 μg/mL (FIG. 5c). The addition of anti-HER2 mAb also improved the killing efficacy of MCAR-Vδ2T cells, with and without IL-15 secretion, against OVCAR3-FG, OVCAR8-FG, and SKOV3-FG cells, whereas no benefit was observed for conventional MCAR-T cells (FIG. 5d-f). We confirmed that CD16^{Lo} Vδ2 T cells lack detectable ADCC functions (FIG. 10c, d). Importantly, ADCC enabled efficient killing of MSLN-negative SKOV3-FG cells by CD16^{Hi} MCAR-Vδ2T cells (FIG. 5f), and this was accompanied by pronounced secretion of IFN-γ (FIG. 5g). Lastly, a repeated tumor challenge assay was performed against ^{KO}OVCAR3-FG cells in the presence or absence of anti-HER2 mAb (FIG. 5h). Antibody treatment greatly enhanced tumor killing by MCAR-and MCAR15-Vδ2T cells, although it was only the MCAR15-Vδ2T cells and antibody combination that established prolonged tumor control (FIG. 5i).

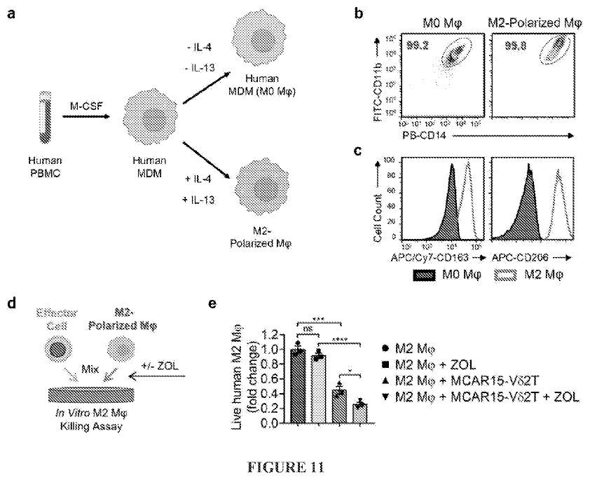

MCAR15-vδ2T cells can kill tumor-associated macrophages in vitro. Macrophages (Mφ) are large, innate immune cells that phagocytose target cells in response to infection or insult and recent work highlights the role macrophages play in both tumor elimination and progression⁵¹⁻⁵⁴. While existing on a continuum, macrophage populations that prevent cancer growth and activate antitumor immunity are commonly referred to as M1-type, and those that promote cancer growth and potentiate immunosuppression as M2-type. Most cancers are primarily populated by M2-type macrophages, which represent a logical target for immunotherapies. We generated human monocyte-derived M2 macrophages by culturing PBMCs in the presence of macrophage colony-stimulating factor (M-CSF), resulting in monocyte-derived macrophages (MDM), followed by culture with IL-4 and IL-13 to produce M2-polarized Mφ (FIG. 11a)⁵⁵. FACS detection of CD11b and CD14 revealed successful production of macrophages and further characterization of M2 Mφ markers CD163 and CD206 confirmed M2 polarization (FIG. 11b, c). MCAR15-Vδ2T cells were cultured with M2 Mφ in vitro and the resulting cytotoxicity was monitored by flow cytometry (FIG. 11d). After a 24 h culture, at a 1:1 MCAR15-γδT: M2 Mφ, 50% of the Mφ cells were killed, and this was increased to 70% upon the addition of ZOL (FIG. 11e). These results indicate that the killing of tumor-associated macrophages (TAMs) may be another mechanism that MCAR15-Vδ2T cells could potentially exploit to mediate antitumor immune reactivity.

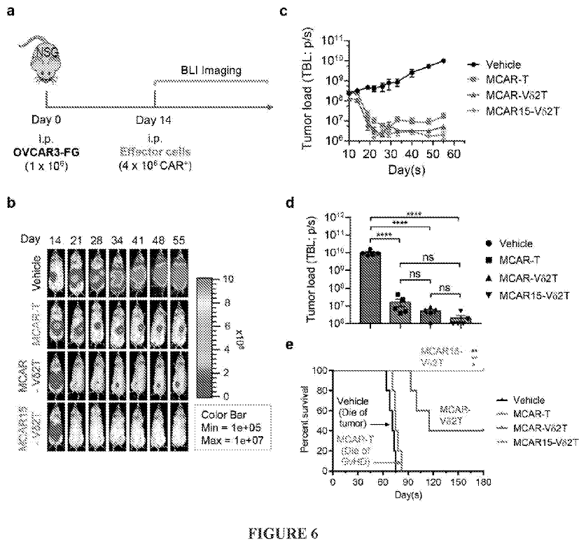

MCAR15-Vδ2T cells are safe and efficacious in intraperitoneal and subcutaneous in vivo ovarian cancer models. The in vivo antitumor activity and safety of MCAR15-Vδ2T cells derived from CD16^{Hi} donors were evaluated in two xenograft tumor models. In the first model, NSG mice were inoculated intraperitoneally (i.p.) with 1×106 OVCAR3-FG cells and fourteen days later the mice were treated with i.p. delivered 4×106 MCAR-T, MCAR-Vδ2T, or MCAR15-Vδ2T cells, or vehicle (PBS) (FIG. 6a). Tumor growth was monitored by live animal imaging using bioluminescence. Within one week of treatment, all the effector cell groups displayed significant tumor retardation compared to the control group and effective tumor control (FIG. 6b-d). Control mice reached endpoint around day 70 post-tumor injection due to cancer burden, and MCAR-T mice perished shortly thereafter due to GvHD (as determined by weight loss, fur loss, and malaise) (FIG. 6e). For mice treated with MCAR-V82T cells, 2/5 mice survived through the end of the study, day 180, with one complete elimination of the tumor and one mouse with tumor relapse, whereas the other three mice succumbed to relapsed tumors. MCAR15-Vδ2T cells resulted in complete remissions in all 5/5 mice through day 180 without any signs of GvHD, highlighting the benefit of IL-15 engineering in long-term intraperitoneal tumor growth inhibition (FIG. 6e).

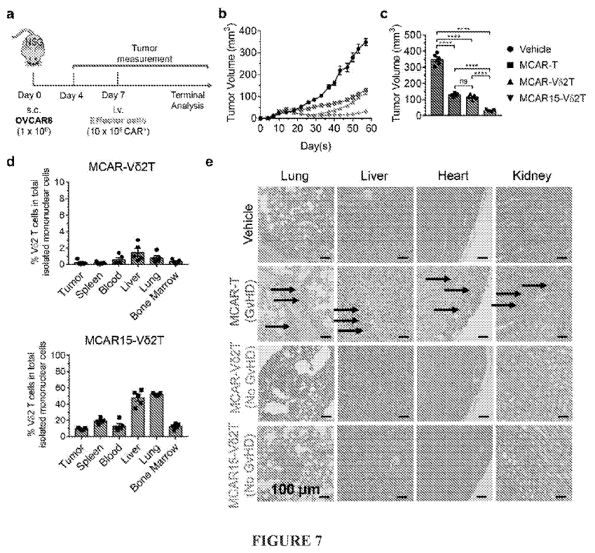

We then tested the therapeutic potential of MCAR15-Vδ2T cells in a subcutaneous tumor model. OVCAR8 subcutaneous tumors were established in NSG mice and, when the tumors reached an average of 50mm³, 10×106 effector cells were administered intravenously (FIG. 7a). All the effector groups resulted in tumor growth inhibition, with MCAR15-Vδ2T cells sustaining significantly enhanced tumor control compared to the all the other groups by Day 57 (FIG. 7b, c). On Day 57, the mice were sacrificed for terminal analysis. Tumors were excised and processed to assess human immune cell infiltration. Mouse tissues were also harvested to assess effector cell persistence throughout the preclinical model as well as xenoreactivity (GvHD). MCAR15-Vδ2T cells displayed robust persistence, as shown by their increased presence in the tumor and all the mouse organs analyzed (FIG. 7d). The superior persistence was not associated with GvHD, as H&E-stained tissue sections collected from experimental mice at day 57 showed MCAR15-Vδ2T cells did not cause the accumulation of mononuclear cell infiltrates in the lung, liver, spleen, nor kidney, whereas MCAR-T cell-treated mice showed distinct manifestations of mononuclear cell aggregates (FIG. 7e). An additional OVCAR8 subcutaneous study showed that CD16^{Hi} MCAR15-Vδ2T cells exhibit superior in vivo tumor control compared to CD16^{Lo} MCAR15-Vδ2T cells (FIG. 11). These in vivo results highlight the potential for CD16^{Hi} MCAR15-Vδ2T cells to treat intraperitoneal solid tumors without causing GvHD. The results also confirm the benefit of engineering CAR-Vδ2 T cells to secrete IL-15.

### METHODS

Mice. NOD. Cg-Prkdc^{SCID}Il2rg^{tm1Wjl}/SzJ (NOD/SCID/IL-2Rγ^{-/-}, NSG) mice were maintained in the animal facilities of the University of California, Los Angeles (UCLA) under the following housing conditions: temperature ranging from 68° F. to 79° F., humidity maintained at 30% to 70%, a light cycle of On at 6:00 am and Off at 6:00 pm, and room pressure set to negative. 6-10 weeks old female mice were used for all experiments unless otherwise indicated. Due to ethical reasons, we terminated experiments when mice developed severe ascites or before tumor volume surpassed 1000 mm³. All animal experiments were approved by the Institutional Animal Care and Use Committee (IACUC) of UCLA. All mice were bred and maintained under specific pathogen-free conditions, and all experiments were conducted in accordance with the animal care and use regulations of the Division of Laboratory Animal Medicine (DLAM) at the UCLA.

Medium, cytokines, and chemicals. Zoledronic acid monohydrate (ZOL) was purchased from Sigma (Cat. SML0223). Recombinant human IL-2, IL-4, IL-7, IL-15,IL-17, interferon gamma (IFN-γ), and tumor necrosis factor alpha (TNF-α) were purchased from PeproTech. RPMI 1640 and DMEM cell culture medium were purchased from Corning Cellgro. Fetal bovine serum (FBS) was purchased from Sigma. Medium supplements, including Penicillin/Streptomycin/Glutamine (P/S/G), MEM non-essential amino acids (NEAA), HEPES Buffer Solution, and Sodium Pyruvate, were purchased from Gibco. Beta-mercaptoethanol (β-ME) was purchased from Sigma. Normocin was purchased from InvivoGen. CryoStor cell cryopreservation media CS10 was purchased from Sigma (Cat. C2874). Complete lymphocyte culture medium (denoted as C10 Medium) was made of RPMI 1640 supplemented with FBS (10% vol/vol), P/S/G (1% vol/vol), MEM NEAA (1% vol/vol), HEPES (10 mM), Sodium Pyruvate (1 mM), β-ME (50 mM), and Normocin (100 mg/mL). The medium for culturing OVCAR3 and OVCAR8 tumor cell line (denoted as R10 medium) was made of RPMI 1640 supplemented with FBS (10% vol/vol) and P/S/G (1% vol/vol). Medium for culturing HEK-293T/17 and SKOV3 tumor cell line (denoted as D10 medium) was made of DMEM supplemented with FBS (10% vol/vol) and P/S/G (1% vol/vol). Freezing medium for cryopreservation of cell lines and PBMC derived cells were made of CryoStor cell cryopreservation media CS10 at a 1:1 ratio with complete base medium.

Cell lines. Human embryonic kidney 293T/17 (HEK-293T/17, ATCC; Cat. CRL-11268), and human ovarian cancer cell lines OVCAR3 (ATCC; Cat. HTB-161), OVCAR8 (NIH; Cat. CVCL_1629), and SKOV3 (ATCC; Cat. HTB-77) were all purchased from American Type Culture Collection (ATCC) or obtained from National Institutes of Health (NIH) under MTA. HEK-293T/17 and SKOV3 cell lines were maintained in D10 medium. OVCAR3 and OVCAR8 cell lines were maintained in R10 medium.

To make stable tumor cell lines overexpressing a firefly luciferase and enhanced green fluorescence protein (FG) dual reporter, the parental tumor cell lines were transduced with a Lenti/FG vector encoding the FG dual reporter⁹². 72 h-post lentivector transduction, cells were subjected to flow cytometry sorting to isolate gene-engineered cells for making stable cell lines. Three FG labeled stable tumor cell lines were generated for this study, including OVCAR3-FG, OVCAR8-FG, and SKOV3-FG.

One additional cell line that overexpresses the FG dual reporter as well as a knockout of mesothelin (MSLN) tumor antigen, ^{KO}OVCAR3-FG, was generated for this study. Briefly, OVCAR3-FG cells were electroporated with a CRISPR-Cas9/MSLN-sgRNA complex composed of pre-mixed Cas9-NLS protein (4 μL at 6.5 μg/μL; UC Berkeley) and MSLN-sgRNA (1 μL at 100 μM). For electroporation, tumor cells were pulsed twice at 1170 V for 30 ms in a Neon Transfection System (Thermo Fisher Scientific; Cat. MPK 5000) following manufacturer's protocol. 72 h post electroporation, the engineered OVCAR3-FG cells were subjected to flow cytometry sorting to isolate MSLN-KO OVCAR3-FG (KOOVCAR3-FG) cell line.

Lentiviral vector construction. Lentiviral vectors used in this study were all constructed from a parental lentivector pMNDW, which contains the MND retroviral LTR U2 region as an internal promoter and contains an additional truncated Woodchuck Responsive Element (WPRE) to stabilize viral mRNA⁹². The 2A sequences derived from porcine teschovirus-1 (P2A), and Thosea asigna virus 2A (T2A) were used to link the inserted genes to achieve co-expression.

The Lenti/FG, Lenti/MCAR, Lenti/MCAR15, and Lenti/CD16 vectors were constructed by inserting into the pMNDW parental vector corresponding the synthetic genes: a bicistronic gene encoding the FG dual reporter, a gene encoding the MSLN-39 targeting CAR (MCAR), a bicistronic gene encoding the same MCAR as well as a secreting form of human IL-15 (MCAR15), and a gene encoding the CD16a (CD16), respectively. The MCAR consists of SS1 scFv, CD8α hinge, CD28 transmembrane domain, CD28 signaling domain, and CD3ζ signaling domain⁹³. The synthetic gene fragments were obtained from GenScript and IDT.

Lentiviruses were produced using HEK-293T/17 cells following a standard transfection protocol. Briefly, HEK-293T/17 cells were co-transfected with three plasmids: a lentiviral vector plasmid, a lentiviral glycoprotein plasmid (pCMV-VSVG), and a lentiviral packaging plasmid (pCMV-Delta R8.9), using TransIT-Lenti Transfection Reagent (Mirus Bio; Cat. MIR 6600) for 16 to 18 hours. This was followed by treatment with 10 mM sodium butyrate for 8 hours. Subsequently, virus-containing supernatants were generated in serum-free UltraCULTURE media (Lonza Walkersville: Cat. BP 12725F) for 48 hours. The supernatants were concentrated using a 100KDa Amicon Ultra-15 Centrifugal Filter Unit (Millipore Sigma; Cat. UFC 910024) at 4000 rcf for 40 minutes at 4° C., and stored as aliquots at −80° C. Lentivector titers were measured by transducing HEK-293T/17 cells with serial dilutions and performing flow cytometry following established protocols.

Antibodies and flow cytometry. Fluorochrome-conjugated antibodies specific for human APC/Cy7-CD45 (Cat. 304014, Clone H130, 1:100 dilution), PE/Cy7-TCRαβ (Cat. 306720, Clone IP26, 1:25 dilution), FITC-CD3 (Cat. 317306, Clone OKT3, 1:200 dilution), PB-CD3 (Cat. 317314, Clone OKT3, 1:100 dilution), FITC-CD27 (Cat. 356403, Clone M-T271, 1:100 dilution), APC/Cy7-CD45RA (Clone HI100, 1:200 dilution), FITC-CD56 (Cat. 318304, Clone HCD56, 1:10 dilution), APC-TCR Vγ9 (Cat. 331309, Clone B3, 1:100 dilution), PerCP-TCR Vδ2 (Cat. 331410, Clone B6, 1:100 dilution), APC-CD16 (Clone 3G 8, 1:250 dilution), PE-CCR2 (Cat. 357206, Clone K036C2, 1:400 dilution), PE/Cy7-CCR4 (Cat. 359410, Clone L291H4, 1:500 dilution), FITC-CCR5 (Cat. 359120, Clone J418F1, 1:200 dilution), PE/Cy7-CXCR3 (Cat. 353720, Clone G025H7, 1:100 dilution), APC/Cy7-IFN-γ (Cat. 502529, Clone B27, 1:100 dilution), FITC-granzyme B (Cat. 372205, Clone QA16A02, 1:1000 dilution), PE/Cy7-perforin (Cat. 308125, Clone dG9, 1:50 dilution), PE-pSTAT5(Tyr694, Cat. 936903, Clone A17016B, 1:50 dilution), PE/Cy7-Bcl-2 (Cat. 633511, Clone BCL/10C4, 1:100 dilution), APC-HER2 (Cat. 324407, Clone 24D2, 1:400 dilution), PB-CD14 (Cat. 301815, Clone 63D3, 1:1000 dilution), FITC-CD11b (Cat. 301330, Clone ICRF44, 1:10000 dilution), APC/Cy7-CD163 (Cat. 333622, Clone GHI/61, 1:500 dilution), APC-CD206 (Cat. 321110, Clone 15-2, 1:500 dilution), and APC-Streptavidin (Cat. 405207, 1:1000 dilution) to bind Biotinylated Human Mesothelin for MCAR staining were all purchased from BioLegend.

Biotinylated Human Mesothelin (Cat. MSN-H82E9, 1:400 dilution) was purchased from ACROBiosystems. Fluorochrome-conjugated antibody specific for human APC-Mesothelin (Cat. FAB 32652A, Clone 420411, 1:100 dilution) was purchased from R&D Systems. Fluorochrome-conjugated antibody specific for human FITC-Bcl-XL (Cat. MA5-28637, Clone 7B2.5, 1:100 dilution) was purchased from Thermo Fisher Scientific. Fluorochrome-conjugated antibody specific for human FITC-TCRγ/δ (Cat. 347903, Clone 11F2, 1:15 dilution) was purchased from BD Biosciences. Fluorochrome-conjugated antibody specific for human PE-TCR Vδ1 (Cat. 130-120-580, Clone REA173, 1:500 dilution) was purchased from Miltenyi Biotec. Fixable Viability Dye eFluor 506 (e506, Cat. 65-0866-18) was purchased from Affymetrix eBioscience. Mouse Fc Block (anti-mouse CD16/32, Cat. 553142, Clone 2.4G2, 1:50 dilution) was purchased from BD Biosciences, and human Fc Receptor Blocking Solution (TrueStain FcX, Cat. 422302, 1:25 dilution) was purchased from BioLegend. InVivoSIM anti-human HER2 (Trastuzumab Biosimilar, Cat. SIM0005) was purchased from BioXCell.

Flow cytometry surface stainings and intracellular stainings were performed following standard protocols, as well as specific instructions provided by a manufacturer for particular antibodies. Intracellular staining of IL15-mediated pro-survival signaling pathway molecules (pSTAT5, Bcl-2, and Bcl-xL) were performed following Foxp3/Transcription Factor Staining protocol (Thermo Fisher Scientific; Cat. 50-112-8857). Stained cells were analyzed using a MACSQuant Analyzer 10 flow cytometer (Miltenyi Biotech). FlowJo software version 10 (BD Biosciences) was used for data analysis.

Enzyme-Linked Immunosorbent Cytokine Assays (ELISA). The ELISA for detecting human IFN-γ was performed following a standard protocol from BD Biosciences. Supernatants from cell culture assays were collected and assayed to quantify IFN-γ. The capture (Cat. 551221, Clone NIB42, 1:250 dilution) and biotinylated (Cat. 554550, Clone 4S. B3, 1:500 dilution) pairs for detecting IFN-γ were purchased from BD Biosciences. The HRP-Avidin conjugate (Cat. 405103, 1:1000 dilution) and the human IFN-γ ELISA standards (Cat. 570209) were purchased from BioLegend. 1-Step™ TMB ELISA Substrate Solutions was purchased from Thermo Fisher Scientific (Cat. 34021). Human IL-15 was quantified with Human IL-15Quantikine ELISA Kit (R&D Systems; Cat. D1500). The samples were analyzed for absorbance at 450 nm using an Infinite M1000 microplate reader (Tecan).

Generation of PBMC-derived conventional αβ T cells and derivatives. Healthy donor human PBMCs were obtained from the UCLA/CFAR Virology Core Laboratory, with identification information removed under federal and state regulations. Protocols using these human cells were exempted by the UCLA Institutional Review Board (IRB), IRB #05-10-093, 21 Jan. 2019. To generate PBMC-derived conventional αβ T (denoted as PBMC-T) cells, 1×10⁶ cells/mL PBMCs were resuspended in C10 medium supplemented with 100 IU/mL human IL-2 (T-medium) and stimulated with 50 ng/ml of the anti-CD3 monoclonal antibody OKT3 (BioLegend; Cat. 317325). 2 days after activation, PBMCs were washed and passaged 3 times per week for 2 weeks to maintain a cell density at 0.5-1×10⁶ cells/mL; fresh T-medium was added at every passage.

To generate MCAR-T cells, 1×10⁶ cells/mL PBMCs were stimulated with 50 ng/ml of the anti-CD 3 monoclonal antibody OKT3 in the T-medium. 2 days after activation of the PBMC cultures, cells were washed, resuspended in the fresh T-medium, and then concentrated MCAR lentivector was added to the PBMC cultures. The following day, transduced cells were washed and passaged 3 times per week for 2 weeks to maintain a cell density at 0.5~1×10⁶ cells/mL; fresh T-medium was added at every passage. The resulting MCAR-T cells were collected and cryopreserved for future use.

Generation of PBMC-derived Vδ2 T cells and derivatives. Healthy donor human PBMCs were obtained from the UCLA/CFAR Virology Core Laboratory, with identification information removed under federal and state regulations. Protocols using these human cells were exempted by the UCLA Institutional Review Board (IRB), IRB #05-10-093, 21 Jan. 2019. To generate PBMC-derived Vδ2 T (denoted as Vδ2T) cells, 2.5×10⁶/mL PBMCs were resuspended in C10 medium supplemented with 100 IU/mL human IL-2 (T-medium) and stimulated with 5 μM ZOL. 3 days after the activation, Vδ2T cells were enriched via TCRγ/δ⁺ T Cell Isolation Kit (Miltenyi Biotech; Cat. 130-092-892), and then resuspended in the fresh T-medium. PBMCs were washed and passaged 3 times per week for 10-14 days to maintain a cell density at 1-1.5×10⁶ cells/mL; fresh T-medium was added at every passage.

To generate MCAR-Vδ2T and MCAR15-Vδ2T cells, 2.5×10⁶/mL PBMCs were resuspended in the T-medium and stimulated with 5 μM ZOL. 3 days after activation, PBMCs were washed, enriched via TCRγ/δ⁺ T Cell Isolation Kit, resuspended in the fresh T-medium, and then concentrated MCAR or MCAR15 lentivector was added to the PBMC cultures. The following day, transduced cells were washed and passaged 3 times per week for 2 weeks to maintain a cell density at 1~1.5×10⁶ cells/mL; fresh T-medium was added at every passage. The resulting MCAR-Vδ2T and MCAR15-Vδ2T cells were collected and cryopreserved for future use.

RNA-Seq analysis of Vδ2T cells. A total of 13 PBMC-derived Vδ2T cell samples were analyzed. Vδ2T cells were expanded and purified according to FIG. 1a. Cell samples were sorted using a FACSAria II flow cytometer (BD Biosciences). Total RNAs were isolated from each cell sample using a miRNeasy Mini Kit (QIAGEN). cDNAs were synthesized using an iScript cDNA Synthesis Kit (BioRad). Libraries were constructed using an Illumina TruSeq Stranded Total RNA Sample Prep kit and sequenced with 50 bp single-end reads (targeting 20×10⁶ reads per sample) on Illumina HiSeq3000 following the manufacturer's instructions and the TCGB Core's standard protocol. The raw sequencing data underwent quality check using FastQC software (version 0.11.9), and fastp (version 0.23.2) was employed to remove sequencing adapters and low-quality bases. The trimmed reads were mapped to the human reference genome (hg38) with STAR 2.7.9a and the gene count matrixes were obtained using featureCounts from Subread package (version 2.0.3). Batch effect removal was carried out using Combat-Seq, which was implemented in the sva package (version 3.44.0). Gene expression counts normalized by sequencing depth (cpm, counts per million) were obtained using edgeR (version 3.38.4). A log-transformation with pseudocount 1 was applied for principal component analysis (PCA) and heatmap generation. The top 500 most variable genes were used for PCA plots. Differential expression analyses were carried out using DESeq2 (version 1.36.0) based on CD16 expression. Gene with exactly 0 p-value was represented as the smallest p-value divide by 10. To control for false discovery rate (FDR), the Benjamini-Hochberg (BH) procedure was applied to adjust p-values, and genes with an adjusted p-value below the 0.05 threshold were identified as differentially expressed. Gene set enrichment analysis was performed based on all genes' differential analysis results using clusterProfiler (version 4.4.4). Gene Ontology over-representation analysis was performed on the differential expressed genes and a Cnet plot was used to display the linkages between differentially expressed genes and the enriched biological processes. The immune cell-type score of each Vδ2 T cell samples were calculated using ssGSEA method based on gene signatures of the 24 immune cell-type from ImmuneCellAI, and the correlations between enrichment scores of the designated immune cell type and the samples' CD16 expression level were plotted using R package corrplot (version 0.92).

In vitro Vδ2T cell phenotype and function analyses. The phenotype of Vδ2T cells and derivatives were studied using flow cytometry, by analyzing cell surface markers including MCAR and MCAR15 expression, memory T cell markers (i.e., CD27 and CD45RA), chemokine receptors (i.e., CXCR3, CCR2, CCR4, and CCR5), and NK receptors (i.e., CD56). The capacity of these cells to produce cytotoxic molecules (i.e., perforin and granzyme B) was studied using flow cytometry via intracellular staining. Intracellular staining of IL15-mediated pro-survival signaling pathway molecules (pSTAT5, Bcl-2, and Bcl-xL) were performed following Foxp3/Transcription Factor Staining protocol. The proliferation of Vδ2T cells was measured by cell counting and flow cytometry (identified as CD3⁺TCR Vδ2⁺) over time.

In vitro 24 h tumor cell killing assay. FG-labeled tumor cells (1×10⁴ cells per well) were co-cultured with effector cells (at indicated ratios) in Corning 96-well clear bottom black plates for 24 h, in C10 medium with or without the addition of ZOL (5 μM). At the end of the culture, live tumor cells were quantified by adding D-luciferin (150 μg/mL; Caliper Life Science) to cell cultures and reading out luciferase activities using an Infinite M1000 microplate reader (Tecan).

In vitro repeated tumor challenge assay. The main FIGS. 4f and 5h illustrate the experimental design employed in this study. Briefly, FG-labeled tumor cells (1×10⁴ cells per well) were co-cultured with effector cells (at indicated ratios) in six Corning 96-well clear bottom black plates containing C10 medium with or without the addition of ZOL (5 μM) or anti-HER2 Ab (0.1 μg/mL). 24 h later, live tumor cells from one plate (1^{st} time point) were quantified by adding D-luciferin to cell cultures and reading out luciferase activities using an Infinite M1000 microplate reader. On day 3, the remaining five plates were centrifuged at 300 rcf for 5 minutes, and the old medium was carefully replaced with fresh C10 medium. Subsequently, the cells from these five plates were resuspended and transferred to five newly seeded tumor cell plates. After 24 h, live tumor cells from one plate (2^{nd} time point) were quantified by measuring luciferase activities. This process was repeated for a total of six time points, spanning 16 days.

In vitro ADCC assay. FG-labeled tumor cells (1×10⁴ cells per well) were seeded and subjected to treatment with anti-HER2 Ab (BioXCell; InVivoSIM anti-human HER2; Cat. SIM0005) at 37° C. for 30 minutes in Corning 96-well clear bottom black plates containing C10 medium. Subsequently, effector cells were added to the antibody treated tumor cells and co-cultured them for 24 h. At the end of the culture, live tumor cells were quantified by adding D-luciferin to cell cultures and reading out luciferase activities using an Infinite M1000 microplate reader (Tecan).

In vitro M2-Polarized Macrophage killing assay. Healthy donor human PBMCs were obtained from the UCLA/CFAR Virology Core Laboratory, with identification information removed under federal and state regulations. Protocols using these human cells were exempted by the UCLA Institutional Review Board (IRB), IRB #05-10-093, 21 Jan. 2019. PBMCs were cultured in serum-free RPMI 1640 media (Corning cellgro, Manassas, VA, USA, #10-040-CV) at 1×10⁷ cells/mL cell density. Subsequently, 10-15 mL of the PBMC suspension was seeded into a 10 cm dish and incubated for 1-2 h in a humidified 37° C., 5% CO2 incubator. Next, the medium containing non-adherent cells was discarded and the dishes were washed twice using PBS. The adherent monocytes were cultured in C10 medium, and human M-CSF (10 ng/ml; PeproTech; Cat. 300-25) for 6 days to generate monocyte-derived macrophages (MDMs). At day 6, the generated MDMs were dissociated by 0.25% Trypsin/EDTA (Gibco; Cat. 25200-056), collected, and reseeded in a 6-well plate in C10 medium at 0.5-1×10⁶ cells/mL for 48 h in the presence of recombinant human IL-4 (10 ng/ml; PeproTech; Cat. 214-14) and human IL-13 (10 ng/ml; PeproTech; Cat. 214-13) to induce MDM polarization. Polarized MDMs were then collected and used for flow cytometry or for setting up in vitro mixed culture experiments.

In vivo bioluminescence live animal imaging (BLI). BLI was performed using a Spectral Advanced Molecular Imaging (AMI) HTX imaging system (Spectral instrument Imaging). Live animal imaging was acquired 5 minutes after intraperitoneal (i.p.) injection of D-Luciferin (1 mg/mouse) for total body bioluminescence. Imaging results were analyzed using the AURA imaging software (Spectral Instrument Imaging).

In vivo antitumor efficacy study in an OVCAR3 human ovarian cancer xenograft NSG mouse model (i.p. tumor inoculation mimicking orthotopic growth of ovarian cancer). The experimental design is shown in the main FIG. 6a. Briefly, on day 0, NSG mice received intraperitoneal (i.p.) inoculation of OVCAR3-FG cells (1×10⁶ cells per mouse). On day 14, the experimental mice were assayed for tumor burden using BLI and then placed into 4 equivalent BLI-expressing groups. On the same day, the experimental mice either received i.p. injection of vehicle (PBS) or effector cells (4×10⁶ CAR+ cells/mouse in PBS). All mice were monitored for survival and their tumor loads were measured twice per week using BLI.

In vivo antitumor efficacy study in an OVCAR8 human ovarian cancer xenograft NSG mouse model (s.c. tumor inoculation mimicking solid tumor growth of ovarian cancer). The experimental design is shown in the main FIG. 7a. Briefly, on day 0, NSG mice received subcutaneous (s.c.) inoculation of OVCAR8 cells (1×10⁶ cells per mouse). On day 7, the experimental mice were assayed for tumor burden and then placed into 4 equivalent tumor size groups. On the same day, the experimental mice either received intravenous (i.v.) injection of vehicle (PBS) or effector cells (10×10⁶ CAR⁺ cells/mouse in PBS). All mice were monitored for survival and their tumor loads were measured twice per week using a Fisherbrand™ Traceable™ digital caliper (Thermo Fisher Scientific). Tumor volume was calculated using the formula: Volume (mm³)=(length×width²)/2. At the end of the experiments, mice were terminated. Solid tumors were retrieved, weighted using a PA84 precision balance (Ohaus), then processed for flow cytometry analysis to detect tumor-infiltrating Vδ2 T cells (identified as hCD45⁺Vδ2⁺ cells). Various mouse tissues (blood, heart, lung, liver, and kidney) were also harvested and processed for flow cytometry analysis to detect tissue biodistribution of the Vδ2 T cells, following established protocols⁹⁴.

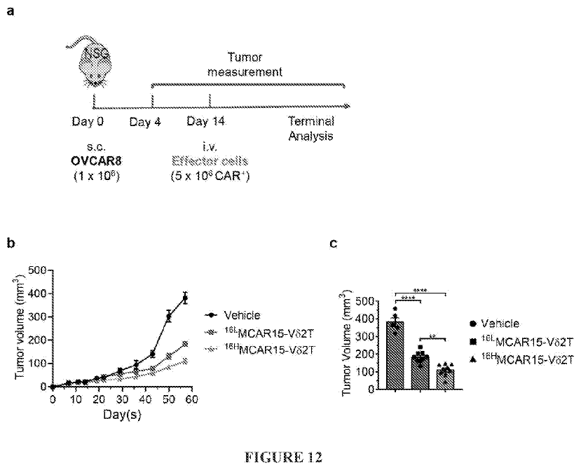

In vivo antitumor efficacy study to compare ^{16H}MCAR15-Vδ2T and ^{16L}MCAR15-Vδ2T cells. The experimental design is shown in FIG. 12a. Briefly, on day 0, NSG mice received subcutaneous (s.c.) inoculation of OVCAR8 cells (1×10⁶ cells per mouse). On day 14, the experimental mice were assayed for tumor burden and then placed into 4 equivalent tumor size groups. On the same day, the experimental mice either received intravenous (i.v.) injection of vehicle (PBS) or effector cells (5×10⁶ CAR⁺ cells/mouse in PBS). All mice were monitored for survival and their tumor loads were measured twice per week using calipers. Tumor volume was calculated using the formula: Volume (mm³)=(length×width²)/2.

### Histopathologic Analysis

Tissues (i.e., spleen, lung, liver, heart, and kidney) were collected from the experimental mice and fixed in 10% neutral buffered formalin for up to 36 h and embedded in paraffin for sectioning (5 μm thickness). Tissue sections were prepared and stained with Hematoxylin and Eosin (H&E) by the UCLA Translational Pathology Core Laboratory, following the Core's standard protocols. Stained sections were imaged using an Olympus BX51 upright microscope equipped with an Optronics Macrofire CCD camera (AU Optronics) at 20× and 40× magnifications. The images were analyzed using Optronics PictureFrame software (AU Optronics).

### Statistics

Rstudio and Graphpad Prism 7 software (Graphpad) were used for statistical data analysis. Student's two-tailed t test was used for pairwise comparisons. Ordinary one-way ANOVA followed by Tukey's or Dunnett's multiple comparisons test was used for multiple comparisons. Log rank (Mantel-Cox) test adjusted for multiple comparisons was used for Meier survival curves analysis. Data are presented as the mean±standard error of the mean (SEM), unless otherwise indicated. In all figures and figure legends, “n” represents the number of samples or animals utilized in the indicated experiments. A P value of less than 0.05 was considered significant. ns, not significant; *p<0.05; **p<0.01; ***p<0.001; ****p<0.0001.

### Data Availability

The bulk RNAseq data sets generated in this study have been deposited in the GEO database under accession code GSE235755.

As noted above, we report the generation and characterization of CAR and IL-15 engineered CD16^{Hi} Vδ2 T cells for the development of allogeneic cellular immunotherapies. CD16 was identified as a biomarker for the selection of Vδ2 T cells with enhanced cytotoxicity. Engineered CD16^{Hi} Vδ2 T cells were generated at high yield and purity, targeted tumors via multiple mechanisms, such as CAR, TCR, and ADCC recognition, and exhibited durable preclinical in vivo tumor control and persistence without signs of GvHD.

To overcome the current challenges of conventional CAR-based αβ T cell therapies, including limited efficacy against solid tumors, the engineering of innate-like and innate immune cells, such γδ T cells, iNKT cells, NK cells, and macrophages, is under active investigation⁵⁶⁻⁵⁹. Analogous to αβ T cells, innate/innate-like cell populations are heterogeneous mixtures of cells with different transcriptional programming, phenotype, and functionality, and certain subsets may be desirable for cell therapy against cancer. Laskowski et al. recently emphasized the inter-donor variability of NK cell profiles and the need for a thorough understanding of NK product characteristics to define biomarkers indicative of greater potency and persistence⁶⁰. Our studies indicate that CD16 can potentially serve as a biomarker for the selection of Vδ2 T cell donors.

Vδ2 T cells are of high interest for developing cancer therapies given their notable safety in the allogeneic setting and intrinsic antitumor functions^{22,61,62}. CAR engineering and altered culture conditions, such as TGF-β supplementation, have been preclinically studied to enhance the therapeutic potential of Vδ2 T cells⁶³⁻⁶⁵, although reported clinical investigation of modified Vδ2 T cells remains scarce⁶². Extensive research on CD16 (FcγRIIIa) and its role in tumor control through ADCC with both therapeutically administered^{47,66,67} and naturally produced⁶⁸ antibodies prompted us to characterize CD16 expression on Vδ2 T cells isolated from healthy human peripheral blood mononuclear cells and assess the potential for CD16 to serve as a biomarker for donor selection. Further support for focusing on CD16 to create cancer immunotherapies comes from several avenues: the development of a high-affinity, non-cleavable CD16 that was incorporated into pluripotent stem cell-derived NK cells for improved antitumor capabilities when combined with mAbs⁶⁹, utilization of CD56^{dim}CD16⁺ NK cells to improve dendritic cell vaccination response⁷⁰, and the bi -and tri-specific killer engagers (BiKEs and TriKEs) against tumor-specific antigens to enhance NK cell-mediated tumor rejection⁷⁴. This data provides evidence that CD16^{Hi} Vδ2 T cells can be another strategy for actualizing the potential of CD16-mediated antitumor immunity.

The expression and functionality of CD16 on Vδ2 T cells have been explored for over two decades²⁷⁻³⁰. One study speculated that the therapeutic potential of PBMC-derived CD16⁺ Vδ2 T cells may be limited by their poor expansion capabilities²⁹ and a single cell RNA-Seq (scRNA-Seq) study indicated PBMC-derived CD16⁺ Vδ2 T cells are fully differentiated⁷⁵, but other researchers demonstrated high yields of CD16⁺ Vδ2 T cells³⁰. In our hands, using traditional ZOL and IL-2 expansion methods, expanded CD16^{Hi} Vδ2 T cells had a similar proliferation and memory status compared to CD16^{Lo} Vδ2 T cells. Vδ2 T cells expanded from CD16^{Hi} donors had heightened cytotoxicity and cytokine production in response to ovarian cancer cell lines and deep RNA-Seq revealed activated, cytotoxic, and phagocytic gene signatures. We further developed CD16^{Hi} Vδ2 T cells for cancer therapy through CAR and IL-15 engineering and confirmed by in vitro and in vivo characterization that CAR15-Vδ2 T cells display robust antitumor efficacy against ovarian cancer models. Although IL-15 injections can increase circulating NK and CD8⁺ T cells, achieving sustained IL-15 signaling using soluble IL-15 is difficult due to its short serum half-life and limited bioavailability⁷⁶. IL-15 self-secretion has the potential to provide sustained and local delivery of IL-15 to engineered immune cells, as well as simplify treatment regimens and reduce systemic toxicity. In ovarian and other solid tumors, MSLN is a promising target, but antigen heterogeneity can curtail the effectiveness of single antigen-targeting modalities. Using CD16^{Hi} MCAR-Vδ2T cells, solid tumors can be killed through CAR-and TCR-mediated recognition as well as combination therapies with HER2 mAbs.

The Vγ9Vδ2 TCR recognizes dysregulated metabolism by binding conformational changes in BTN3A1 that result from altered mevalonate pathways, which commonly occurs in solid tumors and can license Vδ2 T cell killing^{22,77}. Although the ovarian cancer cell lines used in our study required the addition of ZOL for Vδ2 TCR-mediated killing, deregulated cellular energetics is an emerging hallmark of cancer²¹ and metabolic restrictions found in the tumor microenvironment may engender Vδ2 TCR targeting of tumor cells in the absence of exogenous bisphosphonates^{31,32}. Another subset of γδ T cells under evaluation for cancer therapy bears the Vδ1 TCR, and allogeneic CD20-targeting CAR-Vδ1 T cell therapy (ADI-100) has a promising 75% objective response rate (ORR) and 69% complete response (CR) rate in a small Phase I study (n=16) of adults with relapsed/refractory advanced B-cell lymphoma⁷⁸. Importantly, ADI-001 demonstrated a 100% CR rate (n=5) in patients that relapsed after prior autologous anti-CD19 CAR T therapy. Vδ1 T cells mainly recognize glycolipids presented by MHC Class I-like CD1 proteins, which are predominantly expressed by antigen presenting cells⁷⁹⁻⁸¹. Thus, to harness intrinsic TCR recognition of cancer cells, Vδ1 T cells are well-suited for hematological malignancies, whereas Vδ2 T cells can target both liquid and solid cancers. Other potential benefits of the Vδ2 subset include its stimulation by FDA-approved ZOL, higher starting cell number in peripheral blood (1-10% for Vδ2 vs 0.1-1% for Vδ1), and Vδ1 cells do not express CD16⁸². While NK-mediated enhancement of tumor-targeting Abs remains a focus⁸³, our results indicate CD16^{Hi} Vδ2 T cells can be used to achieve antitumor ADCC. Both Vδ1 and Vδ2 subsets exploit NK-like activation and cytotoxicity through various natural killer receptors (NKRs), such as NKG2D and DNAM1, which can enhance the breadth and amplitude of their antitumor activity⁸².

Our exploration into CAR and IL-15 engineered CD16^{Hi} Vδ2 T cell therapy development revealed several areas for further interrogation to determine the potential of this population for cancer treatment, such as T cell memory status, in vivo polarization, immunogenicity, and CD16^{Hi} Vδ2 T cell pool formation. Substantial efforts have focused on creating less differentiated, memory-like cells for CAR-based therapies^{84,85}. Activation and expansion of T cells can result in differentiated final cell products, which can have reduced expansion potential. Interestingly, despite minimal starting numbers, expanded Vδ1 T cells maintain earlier memory status²⁶, which could be a benefit of Vδ1 T cells compared to Vδ2, which in our culture emerge predominantly as T effector memory (TEM) cells. Methods for preserving the memory status of Vδ2 T cells during expansion without compromising expansion rates are under active investigation. Additional characterization, including ex vivo analysis following mouse tumor challenges, can be used to pressure-test the Th1 phenotype of CD16^{Hi} Vδ2 T cell observed in RNA-Seq samples and confirm persistent functionality of the CD16 receptor. Although γδ T cells were identified as a prognostic marker for better outcomes¹⁶ and recently shown to be effectors of immunotherapy in DNA mismatch repair-deficient cancers with HLA class I defects⁸⁶, other studies report the potential negative impact of IL-17-producing γδ T cells³⁴⁻³⁶. CD16^{Hi} Vδ2 T cells express lower levels of ROR1 mRNA than their CD16^{Lo} counterparts and single-sample GSEA indicated CD16 inversely correlates with Th17 polarization, but further assays will be needed to functionally examine Th17 potential in CD16^{Hi} Vδ2 T cells.

It will also be important to address the immunogenicity of engineered CD16^{Hi} Vδ2 T cells. Although lymphodepletion-based preconditioning is standard for adoptive cellular therapy^{87,88}, the question remains whether host-mediated rejection of the infused cells will affect their engraftment and obviate a therapeutic window to elicit meaningful clinical benefit. If immunogenicity were to critically hinder engineered CD16^{Hi} Vδ2 T cells, haploidentical or HLA-matched donors or cloaking strategies, such as HLAI/II knockout and/or HLA-E overexpression, may be necessary to evade immune rejection.

Investigations into how the CD16^{Hi} Vδ2 T cell pool is formed can deepen our understanding of CD16^{Hi} Vδ2 T cells as a unique subpopulation. We have shown that transgenic CD16 on CD16LO Vδ2 T cells did not affect cytotoxicity, supporting the notion that CD16 signifies more than a functional enhancer of Vδ2 T cell tumor killing. It was previously found that FcRγ-deficient NK (g-NK) cells exhibited significantly more robust responsiveness upon stimulation through CD16, and subsequent studies revealed the g-NK cell pool is shaped and maintained in human cytomegalovirus (CMV)-infected individuals by a mechanism that involves both epigenetic modification and antibody-dependent expansion⁸⁹⁻⁹¹. Additional analysis of CD16^{Hi} Vδ2 T cells using scRNA-Seq and epigenomic sequencing may unveil their etiology and engender the creation of further enhanced CD16^{Hi} Vδ2 T cell products.

There are several potential challenges to successful CD16^{Hi} Vδ2 T cell-based therapy. Tumor penetration is an important consideration in developing CAR T cells to treat solid tumors, and while there is preliminary evidence that MCAR15-Vδ2T cells infiltrate subcutaneous tumors in vivo, additional studies and ultimately clinical investigation will be needed to show meaningful tumor penetration in patients. Upon infiltrating the tumor, sufficient persistence and antitumor functionality are significant clinical challenges, as immunorejection and the immunosuppressive tumor microenvironment may thwart CD16^{Hi} Vδ2 T cell-based therapies. Furthermore, although CAR-engineered γδ T cell clinical trials have been conducted and are underway, the large-scale production of MCAR15-Vδ2T cells using CD16^{Hi} donors will require significant manufacturing and process design optimization efforts. The findings from our study highlight the effectiveness of a combination of donor selection based on CD16 expression, CAR engineering, and IL-15 secretion in enhancing the cancer therapy potential of Vδ2 T cells. The high CD16 expression on Vδ2 T cells allows these cells to be used in combination with therapeutic antibodies, while their cytotoxicity towards M2-polarized macrophages provides additional antitumor properties. Given the heterogeneity of solid tumors and their complex immunosuppressive tumor microenvironment, a multi-faceted approach to tumor recognition and immunomodulation will likely be necessary to achieve long-lasting therapeutic results in treatment-resistant patients.

### Example 2: Enhanced Gamma Delta T Cells for Immunotherapy

Cancer T cells are an innate-like subpopulation of T cells that account for 1-5% of peripheral blood mononuclear cells, and Vδ2 T are the most common subset (80-90% of total γδ T cells). Vδ2 T cells do not cause graft-versus-host-disease (GvHD) and possess intrinsic cancer killing abilities, but their clinical application for cancer immunotherapy is limited by their scarcity and persistence, as well as tumor immunosuppression mechanisms.

Chimeric antigen receptor-T (CAR-T) cells have transformed the treatment of hematological malignancies but have not had the same success in combatting solid tumors. Furthermore, all the FDA-approved CAR-T cell therapies are autologous, which is a critical bottleneck in their scalability, affordability, and accessibility, and can cause severe adverse events. To create allogeneic, “off-the-shelf” cell therapies that are both safe and effective for the treatment of liquid and solid tumor types, we have chosen gamma delta (γδ) T cells as an alternative cell source for genetic engineering and cancer treatments. γδ functional readouts indicate CD16 high Vδ2 T (CD16^{Hi} Vδ2 T) cells can exhibit more robust cytotoxicity activity than CD16 low (CD16^{Lo} Vδ2 T) Vδ2 T cells and can perform antibody-dependent cellular cytotoxicity (ADCC).

As discussed below, we have developed a CD16^{Hi} Vδ2 T cell expansion method that results in higher yields than conventional expansion approach. Although experiencing high levels of expansion, the Vδ2 T cells maintain a less differentiated memory-like profile (e.g., memory markers: stem cell-like memory (TSCM): CD27⁺CD45RA⁺; and/or central memory (TCM): CD27⁺CD45RA⁻; and/or effector memory (TEM): CD27⁻CD45RA⁻; and/or terminally differentiated effector memory (TEMRA): CD27⁻CD45RA⁺). Our advancements in Vδ2 T cells may be able to generate potent, high quality Vδ2 cells at scale.

### Illustrative Invention Embodiments

Embodiments of the invention include a donor screening method and a novel expansion approach (Memory Expansion) and associated media, that can be used to generate Vγ9Vδ2 T (referred as Vδ2 T) cells with enhanced antitumor properties.

The donor screening method comprises the selection of donors based on CD16 expression on Vδ2 T cells.

Donor refers to peripheral blood mononuclear cell (PBMC) donor. PBMCs can be isolated from leukopak and used for expansion of Vδ2 T cells.

The expanded CD16^{Hi} Vδ2 T cells can be coupled with monoclonal antibodies to treat different disease targets for combination therapy.

The Memory Expansion includes 1) initial stimulation of Vδ2 T cells from PBMCs; 2) in vitro expansion of Vδ2 T cells using a Memory Medium disclosed herein. This expansion method can be used to expand all types of Vδ2 T cells, including populations of CD16^{Hi} Vδ2 T cells.

The resulting memory-like Vδ2 T (^{Mem}Vδ2 T) cells generated using Memory Expansion possess central memory (T_{CM}) and stem cell-like memory (T_{SCM}) phenotype, and have better self-renewal potential and persistence than Vδ2 T cells expanded by conventional culture approach.

In addition to the screening and the expansion method, the cultured Vδ2 T cells can be gene-engineered (e.g., gene overexpression, gene knock-down/knock-out, gene disruption) to generate Vδ2 T cell derivatives with enhanced therapeutic potential. See examples at the below “Vδ2 T cell derivatives” section.

### ILLUSTRATIVE METHODS AND MEDIAS

### Donor Screening

Donors are screened for CD16 expression on their PBMC Vδ2 T cells. CD16 high donors are selected as PBMC donors.

### Memory-Like Vδ2 T Cell Culture

PBMCs are cultured to generate memory-like Vδ2 T cells. PBMCs can be obtained from the selected CD16 high donors.

Vδ2 T cells are stimulated and expanded by supplementing into the cell culture Vδ2-Stimulatory Reagents, including but not limited to, TCR cognate antigens, phosphoantigens, small molecules, and/or antibodies. Examples of such reagents include isopentenyl pyrophosphate, zoledronate, pamidronate, risedronate, alendronate, ibandronate, tiludronate, etidronate, anti-γδTCR antibodies, non-specific TCR stimulatory reagents (anti-CD3/anti-CD28 antibodies or antibody-coated beads, Concanavalin A, PMA/Ionomycin, and artificial APCs), anti-CD16 antibodies, and others.

Memory-like feature of the cultured Vδ2 T cells can be achieved by supplementing into the cell culture Memory-Promoting Reagents, including but not limited to, serum albumin, L-ascorbic acid, 2-mercaptoethanol, IL-2, IL-4, IL-7, IL-12,IL-15, IL-18, IL-21, IL-23, TNFα, SDF-1α, TGF-β, and Wnt activators or glycogen synthase kinase-3 (GSK-3) inhibitors such as Wnt3A, CHIR99021, AR-A014418, TWS119, LY2090314, 9-ING-41, lithium chloride (LiCl), BIO (6-bromoindirubin-3-oxime, 6-Bromoindirubin-3′-oxime). The Memory Medium may also include tyrosine kinase inhibitors such as dasatinib, ibrutinib, acalabrutinib, zanubrutinib, and others. The Memory Medium can be used to culture and expand all types of Vδ2 T cells, including CD16^{Hi} Vδ2 T cells.

The cell culture base media can be, including but not limited to, CTS OpTmizer, TexMACS, RPMI, DMEM, X-Vivo15, and others. The cell culture approach can be serum-free and feeder-free.

CD16^{Hi} Vδ2 T cells can be enriched by magnetic bead sorting or fluorescence-activated cell sorting (FACS) based on CD16 expression at any stages of the culture (e.g., prior to, during process, or after expansion of Vδ2 T cells).

The resulting CD16^{Hi} Vδ2 T cell products can be cryopreserved and stored for off-the-shelf distribution.

### Vδ2 T Cell Derivatives

In some embodiments, the Vδ2 T cells can be engineered to express transgenes. In one embodiment, such transgenes encode disease targeting molecules such as chimeric antigen receptors (CARs), and other native or synthetic receptor/ligands. In another embodiment, such transgenes can encode T cell regulatory proteins such as IL-2, IL-7, IL-15, IFN-γ, TNFα, CD28, 4-1BB, OX40, ICOS, FOXP3, and others. Transgenes can be introduced at various culture stages.

In some embodiments, the Vδ2 T cells can be further engineered to disrupt selected genes using gene editing tools (CRISPR, TALEN, Zinc-Finger, and others). In one embodiment, disrupted genes encode T cell immune checkpoint inhibitors (PD-1, CTLA-4, TIM-3, LAG-3, and others). Deficiency of these negative regulatory genes may enhance the disease fighting capacity of Vδ2 T cells, making them resistance to disease-induced anergy and tolerance.

In some embodiments, Vδ2 T cells can be further engineered to make them suitable for allogeneic adoptive transfer, thereby suitable for serving as off-the-shelf cellular products. In one embodiment, genes encoding MHC molecules or MHC expression/display regulatory molecules [MHC molecules, B2M, CIITA (Class II transcription activator control induction of MHC class II mRNA expression), and others]. Lack of MHC molecule expression on Vδ2 T cells makes them resistant to allogeneic host T cell-mediated depletion. In another embodiment, MHC class-I deficient Vδ2 T cells will be further engineered to overexpress an HLA-E gene that will endow them resistant to host NK cell-mediated depletion.

### Novel Features and Advantages of Embodiments of the Invention

When compared to the conventional method of generating Vδ2 T cell products for cancer immunotherapy, this invention offers a PBMC donor screening method and a cell culture approach that can generate Vδ2 T cells with enhanced antitumor properties. Unique features of this invention include:
1) It selects donors who possess high-performance Vδ2 T cells.
2) The resulting Vδ2 T cells show strong anti-tumour cytotoxicity, and are less likely to polarize into pro-tumoral or pathologic (e.g., Th17-like) γδ T cells in patients.
3) The resulting Vδ2 T cells possess ADCC function and can be coupled with monoclonal antibodies for combination therapy. This offers an opportunity to overcome tumor immune evasion in cancer therapy.
4) The resulting Vδ2 T cells are of higher yield! From a single PBMC leukopak, over 10¹¹ high-performance memory-like Vδ2 T cells can be produced, that can potentially be formulated into 100-1,000 doses (estimated based on the approved CAR-T cell therapy dose at 10⁸-10⁹ cells per dose).
5) The resulting Vδ2 T cells are of central memory (T_{CM}) and stem cell memory (T_{SCM}) phenotype, and show strong persistence and self-renewal potential.
6) The resulting Vδ2 T cells can be effectively gene-engineered to produce immune-enhanced Vδ2 T cell products and derivatives!

Donor screening and robust generation of CD16^{Hi} memory-like Vδ2 T cells of high yield are demonstrated. Such Vδ2 T cells can be effectively engineered to express tumor targeting molecules (e.g., CAR) and immune enhancement molecules (e.g., IL-15), without compromising the yields. Ex vivo and in vivo studies are performed, showing high safety, high antitumor efficacy, high durability, and multiple tumor-targeting mechanisms to overcome tumor immune evasions.

### REFERENCES

1. Westin, J. R. et al. Efficacy and safety of CD19-directed CAR-T cell therapies in patients with relapsed/refractory aggressive B-cell lymphomas: Observations from the JULIET, ZUMA-1, and TRANSCEND trials. Am J Hematol. 96, 1295-1312 (2021).

2. Munshi, N. C. et al. Idecabtagene Vicleucel in Relapsed and Refractory Multiple Myeloma. N Engl J Med. 384, 705-716 (2021).

3. Berdeja, J. G. et al. Ciltacabtagene autoleucel, a B-cell maturation antigen-directed chimeric antigen receptor T-cell therapy in patients with relapsed or refractory multiple myeloma (CARTITUDE-1): a phase 1b/2 open-label study. Lancet. 398, 314-324 (2021).

4. Larson, R. C. & Maus, M. V. Recent advances and discoveries in the mechanisms and functions of CAR T cells. Nat Rev Cancer. 21, 145-161 (2021).

5. Rafiq, S., Hackett, C. S. & Brentjens, R. J. Engineering strategies to overcome the current roadblocks in CAR T cell therapy. Nat Rev Clin Oncol. 17, 147-167 (2020).

6. Majzner, R. G. et al. GD2-CAR T cell therapy for H3K27M-mutated diffuse midline gliomas. Nature. 603, 934-941 (2022).

7. Qi, C. et al. Claudin18.2-specific CAR T cells in gastrointestinal cancers: phase 1 trial interim results. Nat Med. 28, 1189-1198 (2022).

8. Marofi, F. et al. CAR T cells in solid tumors: challenges and opportunities. Stem Cell Res Ther. 12, 81(2021 ).

9. Sterner, R. C. & Sterner, R. M. CAR-T cell therapy: current limitations and potential strategies. Blood Cancer J. 11, (2021).

10. Caldwell, K. J., Gottschalk, S. & Talleur, A. C. Allogeneic CAR Cell Therapy—More Than a Pipe Dream. Front Immunol. 11, 618427(2021 ).

11. Cortés-Selva, D., Dasgupta, B., Singh, S. & Grewal, I. S. Innate and Innate-Like Cells: The Future of Chimeric Antigen Receptor (CAR) Cell Therapy. Trends Pharmacol Sci. 42, 45-59 (2021).

12. Li, Y.-R., Dunn, Z. S., Zhou, Y., Lee, D. & Yang, L. Development of Stem Cell-Derived Immune Cells for Off-the-Shelf Cancer Immunotherapies. Cells. 10, 12 (2021).

13. Hayday, A. C. γδ T Cell Update: Adaptate Orchestrators of Immune Surveillance. J Immunol. 203, 311-320 (2019).

14. Ferreira, L. M. R. Gammadelta T Cells: Innately Adaptive Immune Cells? Int Rev Immunol. 32, 223-248 (2013).

15. Correia, D. V, Lopes, A. & Silva-Santos, B. Tumor cell recognition by γδ T lymphocytes: T-cell receptor vs. NK-cell receptors. Oncoimmunology. 2, e22892 (2013).

16. Gentles, A. J. et al. The prognostic landscape of genes and infiltrating immune cells across human cancers. Nat Med. 21, 938-945 (2015).

17. Jhita, N. & Raikar, S. S. Allogeneic gamma delta T cells as adoptive cellular therapy for hematologic malignancies. Explor Immunol. 2, 334-350 (2022).

18. Herrmann, T., Fichtner, A. S. & Karunakaran, M. M. An Update on the Molecular Basis of Phosphoantigen Recognition by Vγ9Vδ2 T Cells. Cells. 9, 6 (2020).

19. Karunakaran, M. M. et al. Butyrophilin-2A1 Directly Binds Germline-Encoded Regions of the Vγ9Vδ2 TCR and Is Essential for Phosphoantigen Sensing. Immunity. 52, 3 (2020).

20. Rigau, M. et al. Butyrophilin 2A1 is essential for phosphoantigen reactivity by γδ T cells. Science. 367, 6478(2020 ).

21. Hanahan, D. & Weinberg, R. A. Hallmarks of Cancer: The Next Generation. Cell. 144, 646-674 (2011).

22. Hoeres, T., Smetak, M., Pretscher, D. & Wilhelm, M. Improving the Efficiency of Vγ9Vδ2 T-Cell Immunotherapy in Cancer. Front Immunol. 9, 800(2018 ).

23. Liu, E. et al. Cord blood NK cells engineered to express IL-15 and a CD19-targeted CAR show long-term persistence and potent antitumor activity. Leukemia 32, 520-531 (2018).

24. Goodridge, J. et al. Abstract 3207: Preclinical development of first-of-kind dual-targeted off-the-shelf CAR-NK cell product with engineered persistence for an effective treatment of B cell malignancies. Cancer Res. 79, 3207(2019 ).

25. Xu, X. et al. NKT Cells Coexpressing a GD2-Specific Chimeric Antigen Receptor and IL15 Show Enhanced In Vivo Persistence and Antitumor Activity against Neuroblastoma. Clin Cancer Res. 25, 7126-7138 (2019).

26. Makkouk, A. et al. Off-the-shelf Vδ1 gamma delta T cells engineered with glypican-3 (GPC-3)-specific chimeric antigen receptor (CAR) and soluble IL-15 display robust antitumor efficacy against hepatocellular carcinoma. J Immunother Cancer. 9, 12 (2021).

27. Braakman, E., van de Winkel, J. G. J., van Krimpen, B. A., Jansze, M. & Bolhuis, R. L. H. CD16 on human γδ T lymphocytes: Expression, function, and specificity for mouse IgG isotypes. Cell Immunol. 143, 97-107 (1992).

28. Lafont, V., Liautard, J., Liautard, J. P. & Favero, J. Production of TNF-alpha by human V gamma 9V delta 2 T cells via engagement of Fc gamma RIIIA, the low affinity type 3 receptor for the Fc portion of IgG, expressed upon TCR activation by nonpeptidic antigen. J Immunol. 166, 7190-7199 (2001).

29. Angelini, D. F. et al. FcgammaRIII discriminates between 2 subsets of Vgamma9Vdelta2 effector cells with different responses and activation pathways. Blood. 104, 1801-1807 (2004).

30. Tokuyama, H. et al. Vγ9Vδ2 T cell cytotoxicity against tumor cells is enhanced by monoclonal antibody drugs-Rituximab and trastuzumab. Int J Cancer. 122, 2526-2534 (2008).

31. Gober, H.-J. et al. Human T cell receptor gammadelta cells recognize endogenous mevalonate metabolites in tumor cells. J Exp Med. 197, 163-168 (2003).

32. Mattarollo, S. R., Kenna, T., Nieda, M. & Nicol, A. J. Chemotherapy and zoledronate sensitize solid tumour cells to Vγ9Vδ2 T cell cytotoxicity. Cancer Immunol Immunother. 56, 1285-1297 (2007).

33. Jing, Y. et al. Identification of an ADAM17 cleavage region in human CD16 (FcγRIII) and the engineering of a non-cleavable version of the receptor in NK cells. PLoS One. 10, e0121788 (2015).

34. Wakita, D. et al. Tumor-infiltrating IL-17-producing γδ T cells support the progression of tumor by promoting angiogenesis. Eur J Immunol. 40, 1927-1937 (2010).

35. Coffelt, S. B. et al. IL-17-producing γδ T cells and neutrophils conspire to promote breast cancer metastasis. Nature. 522, 345-348 (2015).

36. Agerholm, R. & Bekiaris, V. Evolved to protect, designed to destroy: IL-17-producing γδ T cells in infection, inflammation, and cancer. Eur J Immunol. 51, 2164-2177 (2021).

37. Love, M. I., Huber, W. & Anders, S. Moderated estimation of fold change and dispersion for RNA-seq data with DESeq2. Genome Biol. 15, 550(2014).

38. Yu, G., Wang, L. G., Han, Y. & He, Q. Y. ClusterProfiler: An R package for comparing biological themes among gene clusters. Omi A J Integr Biol. 16, 284-287 (2012).

39. Miao, Y.-R. et al. ImmuCellAI: A Unique Method for Comprehensive T-Cell Subsets Abundance Prediction and its Application in Cancer Immunotherapy. Adv Sci. 7, 1902880(2020 ).

40. Hänzelmann, S., Castelo, R. & Guinney, J. GSVA: gene set variation analysis for microarray and RNA-Seq data. BMC Bioinformatics. 14, 7 (2013).

41. Morello, A., Sadelain, M. & Adusumilli, P. S. Mesothelin-Targeted CARs:

Driving T Cells to Solid Tumors. Cancer Discov. 6, 133-146 (2016).

42. Hassan, R. et al. Mesothelin Immunotherapy for Cancer: Ready for Prime Time? J Clin Oncol. 34, 4171-4179 (2016).

43. Hoyos, V. et al. Engineering CD19-specific T lymphocytes with interleukin-15 and a suicide gene to enhance their anti-lymphoma/leukemia effects and safety. Leukemia. 24, 1160-1170 (2010).

44. Dieli, F. et al. Differentiation of effector/memory Vdelta2 T cells and migratory routes in lymph nodes or inflammatory sites. J Exp Med. 198, 391-62 397(2003 ).

45. Comeau, K., Paradis, P. & Schiffrin, E. L. Human and murine memory γδ T cells: Evidence for acquired immune memory in bacterial and viral infections and autoimmunity. Cell Immunol. 357, 104217(2020 ).

46. Rajasekaran, N., Chester, C., Yonezawa, A., Zhao, X. & Kohrt, H. E. Enhancement of antibody-dependent cell mediated cytotoxicity: a new era in cancer treatment. ImmunoTargets Ther. 4, 91-100 (2015).

47. Graziano, R. F. & Engelhardt, J. J. Role of FcγRs in Antibody-Based Cancer Therapy. Curr Top Microbiol Immunol. 423, 13-34 (2019).

48. Hayes, J. M., Wormald, M. R., Rudd, P. M. & Davey, G. P. Fc gamma receptors: glycobiology and therapeutic prospects. J Inflamm Res. 9, 209-219 (2016).

49. Wallace, P. K., Howell, A. L. & Fanger, M. W. Role of Fcγ receptors in cancer and infectious disease. J Leukoc Biol. 55, 816-826 (1994).

50. Zahavi, D., AlDeghaither, D., O'Connell, A. & Weiner, L. M. Enhancing antibody-dependent cell-mediated cytotoxicity: a strategy for improving antibody-based immunotherapy. Antib Ther. 1, 7-12 (2018).

51. Ostuni, R., Kratochvill, F., Murray, P. J. & Natoli, G. Macrophages and cancer: from mechanisms to therapeutic implications. Trends Immunol. 36, 229-239 (2015).

52. Brown, J. M., Recht, L. & Strober, S. The Promise of Targeting Macrophages in Cancer Therapy. Clin Cancer Res. 23, 3241-3250 (2017).

53. Poh, A. R. & Ernst, M. Targeting Macrophages in Cancer: From Bench to Bedside. Front Oncol. 8, 49(2018 ).

54. Pathria, P., Louis, T. L. & Varner, J. A. Targeting Tumor-Associated Macrophages in Cancer. Trends Immunol. 40, 310-327 (2019).

55. Li, Y.-R. et al. Targeting Immunosuppressive Tumor-Associated Macrophages Using Innate T Cells for Enhanced Antitumor Reactivity. Cancers. 14, 11 (2022).

56. Rezvani, K. Adoptive cell therapy using engineered natural killer cells. Bone Marrow Transplant. 54, 785-788 (2019).

57. Anderson, N. R., Minutolo, N. G., Gill, S. & Klichinsky, M. Macrophage-Based Approaches for Cancer Immunotherapy. Cancer Res. 81, 1201-1208 (2021).

58. Myers, J. A. & Miller, J. S. Exploring the NK cell platform for cancer immunotherapy. Nat Rev Clin Oncol. 18, 85-100 (2021).

59. Molgora, M. & Colonna, M. Innate-like T cells: A promising asset in anti-cancer immunity. Cancer Cell. 40, 714-716 (2022).

60. Laskowski, T. J., Biederstädt, A. & Rezvani, K. Natural killer cells in antitumour adoptive cell immunotherapy. Nat Rev Cancer. 22, 557-575 (2022).

61. Rafia, C. et al. Beyond CAR T cells: Engineered Vγ9Vδ2 T cells to fight solid tumors. Immunol Rev. 298, 117-133 (2020).

62. Lee, D. et al. Human γδ T Cell Subsets and Their Clinical Applications for Cancer Immunotherapy. Cancers. 14, 12 (2022).

63. Capsomidis, A. et al. Chimeric Antigen Receptor-Engineered Human Gamma Delta T Cells: Enhanced Cytotoxicity with Retention of Cross Presentation. Mol Ther. 26, 354-365 (2018).

64. Rozenbaum, M. et al. Gamma-Delta CAR-T Cells Show CAR-Directed and Independent Activity Against Leukemia. Front Immunol. 11, 1347(2020 ).

65. Beatson, R. E. et al. TGF-β1 potentiates Vγ9Vδ2 T cell adoptive immunotherapy of cancer. Cell Rep Med. 2, 12 (2021).

66. Iannello, A. et al. Role of antibody-dependent cell-mediated cytotoxicity in the efficacy of therapeutic anti-cancer monoclonal antibodies. Cancer Metastasis Rev. 24, 487-499 (2005).

67. Natsume, A., Niwa, R. & Satoh, M. Improving effector functions of antibodies for cancer treatment: Enhancing ADCC and CDC. Drug Des Devel Ther. 3, 7-16 (2009).

68. Rawat, K., Tewari, A., Morrisson, M. J., Wager, T. D. & Jakubzick, C. V. Redefining innate natural antibodies as important contributors to anti-tumor immunity. Elife. 10, e69713 (2021).

69. Zhu, H. et al. Pluripotent stem cell-derived NK cells with high-affinity noncleavable CD16a mediate improved antitumor activity. Blood. 135, 399-410 (2020).

70. Lion, E., Smits, E. L. J. M., Berneman, Z. N. & Van Tendeloo, V. F. I. NK Cells: Key to Success of DC-Based Cancer Vaccines? Oncologist. 17, 1256-1270 (2012).

71. Hartmann, F. et al. Treatment of Refractory Hodgkin's Disease With an Anti-CD16/CD30 Bispecific Antibody. Blood. 89, 2042-2047 (1997).

72. Asano, R. et al. Construction and humanization of a functional bispecific EGFR×CD16 diabody using a refolding system. FEBS J. 279, 223-233 (2012).

73. Ochi, F. et al. Gene-Modified Human α/β-T Cells Expressing a Chimeric CD16-CD3ζ Receptor as Adoptively Transferable Effector Cells for Anticancer Monoclonal Antibody Therapy. Cancer Immunol Res. 2, 249-262 (2014).

74. Davis, Z. B., Vallera, D. A., Miller, J. S. & Felices, M. Natural killer cells unleashed: Checkpoint receptor blockade and BiKE/TriKE utilization in NK-mediated anti-tumor immunotherapy. Semin Immunol. 31, 64-75 (2017).

75. Pizzolato, G., Kaminski, H., Tosolini, M. & Franchini, D. Single-cell RNA sequencing unveils the shared and the distinct cytotoxic hallmarks of human TCRV δ 1 and TCRV δ2 γδ T lymphocytes. Proc Natl Acad Sci U S A. 116, 24 (2019).

76. Zhou, Y. et al. Interleukin 15 in Cell-Based Cancer Immunotherapy. Int J Mol Sci. 23, 13 (2022).

77. Lai, A. Y. et al. Cutting Edge: Bispecific γδ T Cell Engager Containing Heterodimeric BTN2A1 and BTN3A1 Promotes Targeted Activation of Vγ9Vδ2(+) T Cells in the Presence of Costimulation by CD28 or NKG2D. J Immunol. 209, 8 (2022).

78. Neelapu, S. S. et al. A phase 1 study of ADI-001: Anti-CD20 CAR-engineered allogeneic gamma delta (γδ) T cells in adults with B-cell malignancies. J Clin Oncol. 40, 7509(2022 ).

79. Luoma, A. M., Castro, C. D. & Adams, E. J. γδ T cell surveillance via CD1 molecules. Trends Immunol. 35, 613-621 (2014).

80. Bai, L. et al. The majority of CD1d-sulfatide-specific T cells in human blood use a semiinvariant Vδ1 TCR. Eur J Immunol. 42, 2505-2510 (2012).

81. Ulrichs, T. & Porcelli, S. A. CD1 proteins: targets of T cell recognition in innate and adaptive immunity. Rev Immunogenet. 2, 416-432 (2000).

82. Mensurado, S., Blanco-domínguez, R. & Silva-santos, B. The emerging roles of γδ T cells in cancer immunotherapy. Nat Rev Clin Oncol. 20, 178-191 (2023).

83. Capuano, C. et al. Harnessing CD16-Mediated NK Cell Functions to Enhance Therapeutic Efficacy of Tumor-Targeting mAbs. Cancers. 13, 10 (2021).

84. Kawalekar, O. U. et al. Distinct Signaling of Coreceptors Regulates Specific Metabolism Pathways and Impacts Memory Development in CAR T Cells. Immunity. 44, 380-390 (2016).

85. Milone, M. C. et al. Engineering-enhanced CAR T cells for improved cancer therapy. Nat Cancer. 2, 780-793 (2021).

86. de Vries, N. L. et al. γδ T cells are effectors of immunotherapy in cancers with HLA class I defects. Nature. 613, 743-750 (2023).

87. Dudley, M. E. et al. Adoptive cell therapy for patients with metastatic melanoma: evaluation of intensive myeloablative chemoradiation preparative regimens. J Clin Oncol. 26, 5233-5239 (2008).

88. Amini, L. et al. Preparing for CAR T cell therapy: patient selection, bridging therapies and lymphodepletion. Nat Rev Clin Oncol. 19, 342-355 (2022).

89. Hwang, I. et al. Identification of human NK cells that are deficient for signaling adaptor FcRγ and specialized for antibody-dependent immune functions. Int Immunol. 24, 793-802 (2012).

90. Lee, J. et al. Epigenetic Modification and Antibody-Dependent Expansion of Memory-like NK Cells in Human Cytomegalovirus-Infected Individuals. Immunity. 42, 431-442 (2015).

91. Bigley, A. B. et al. FcϵRIγ-negative NK cells persist in vivo and enhance efficacy of therapeutic monoclonal antibodies in multiple myeloma. Blood Adv. 5, 3021-3031 (2021).

92. Li, Y. et al. Development of allogeneic HSC-engineered iNKT cells for off-the-shelf cancer immunotherapy. Cell Rep Med. 2, 11(2021 ).

93. Carpenito, C. et al. Control of large, established tumor xenografts with genetically retargeted human T cells containing CD28 and CD137 domains. Proc Natl Acad Sci U S A. 106, 9 (2009).

94. Smith, D. J. et al. Genetic engineering of hematopoietic stem cells to generate invariant natural killer T cells. Proc Natl Acad Sci U S A. 112, 1523-1528 (2015).

### Additional Publications

Lafont, V., Liautard, J., Liautard, J. P., & Favero, J. (2001). Production of TNF-alpha by human V gamma 9V delta 2 T cells via engagement of Fc gamma RIIIA, the low affinity type 3 receptor for the Fc portion of IgG, expressed upon TCR activation by nonpeptidic antigen. Journal of immunology (Baltimore, Md.: 1950), 166(12), 7190-7199. https://doi.org/10.4049/jimmunol.166.12.7190

Angelini, D. F., Borsellino, G., Poupot, M., Diamantini, A., Poupot, R., Bernardi, G., Poccia, F., Fournié, J. J., & Battistini, L. (2004). FcgammaRIII discriminates between 2 subsets of Vgamma9Vdelta2 effector cells with different responses and activation pathways. Blood, 104(6), 1801-1807. https://doi.org/10.1182/blood-2004-01-0331

Tokuyama, H., Hagi, T., Mattarollo, S. R., Morley, J., Wang, Q., So, H. F., Moriyasu, F., Nieda, M., & Nicol, A. J. (2008). V gamma 9 V delta 2 T cell cytotoxicity against tumor cells is enhanced by monoclonal antibody drugs—rituximab and trastuzumab. International journal of cancer, 122(11), 2526-2534.https://doi.org/10.1002/ijc.23365

Gattinoni, L., Zhong, X. S., Palmer, D. C., Ji, Y., Hinrichs, C. S., Yu, Z., Wrzesinski, C., Boni, A., Cassard, L., Garvin, L. M., Paulos, C. M., Muranski, P., & Restifo, N. P. (2009). Wnt signaling arrests effector T cell differentiation and generates CD8+ memory stem cells. Nature medicine, 15(7), 808-813.https://doi.org/10.1038/nm.1982

Lee, D., Rosenthal, C. J., Penn, N. E., Dunn, Z. S., Zhou, Y., & Yang, L. (2022). Human γδ T Cell Subsets and Their Clinical Applications for Cancer Immunotherapy. Cancers, 14(12), 3005; and Silva-Santos, B., Serre, K., & Norell, H. (2015). γδ T cells in cancer. Nature reviews. Immunology, 15(11), 683-691.

All publications mentioned herein (e.g., U.S. Pat. No. 10,316,289; U.S. Pat. No. 11,111,478; WO2009083755; PCT Published International Application Nos. PCT/US21/65349; PCT/US19/36786 and. PCT/US2020/037486; U.S. patent application Ser. No. 15/320,037; as well as Zarin et al., Cell Immunol. 2015 Jul;296(1):70-5. doi: 10.1016/j.cellimm.2015.03.007. Epub 2015, those listed above etc.) are incorporated by reference to disclose and describe aspects, methods and/or materials in connection with the cited publications. Many of the techniques and procedures described or referenced herein are well understood and commonly employed by those skilled in the art.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

1. A method of growing mammalian cells, the method comprising:
obtaining a population of lymphocytes;
identifying, selecting and/or purifying cells within the population of T lymphocytes that express CD16 in combination with Vδ2; and
expanding the T lymphocytes that express CD16 in combination with Vδ2.

2. The method of claim 1, wherein the selected and/or purified T lymphocytes that express CD16 in combination with Vδ2 are expanded by disposing the T lymphocytes in a cell culture media comprising a combination of γδ T lymphocyte stimulators, a combination of cytokines, a combination of Wnt activators and GSK-3B inhibitors, and/or a combination of tyrosine kinase inhibitors.

3. The method of claim 2, wherein:
T lymphocyte stimulators comprise a bisphosphonate;
cytokines comprise IL-2, IL-7, IL-15, and IL-21;
GSK-3β inhibitors comprise TWS119; and/or
tyrosine kinase inhibitors comprises dasatinib, ibrutinib, acalabrutinib, or zanubrutinib.

4. The method of claim 1, wherein the population of lymphocytes is selected from one or more individuals identified as having at least 35% CD16 positive cells within the population of Vδ2 T lymphocytes obtained from the individual.

5. The method of claim 1, wherein the population of lymphocytes is selected from one or more individuals identified as having less than 35% CD16 positive cells within the population of Vδ2 T lymphocytes obtained from the individual.

6. The method of claim 1, wherein the method includes examining cells within the population of lymphocytes in a cellular cytotoxicity assay.

7. The method of claim 1, wherein the population of lymphocytes are obtained from the peripheral blood or cord blood of one or more donors.

8. The method of claim 1, wherein:
the T lymphocytes are expanded by at least 1,000, 2,000, 3,000, 4,000, 5,000, 6,000, 7,000, 8,000, 9,000, and 10,000 fold; and/or
the T lymphocytes are expanded in cell culture for over one month and/or restimulated while maintaining a less differentiated memory status.

9. The method of claim 8, further comprising cryopreserving the expanded cells.

10. A method of making a cell culture media for expanding T lymphocytes that express CD16 in combination with Vδ2, the method comprising disposing together a combination of γδ T lymphocyte stimulators, a combination of cytokines, a combination of Wnt activators; a combination of GSK-3β inhibitors, and/or a combination of tyrosine kinase inhibitors.

11. The method of claim 10, wherein:
T lymphocyte stimulators comprise a bisphosphonate;
Cytokines comprise IL-2, IL-7, IL-15, and IL-21;
GSK-3β inhibitors comprise TWS119; and/or
a tyrosine kinase inhibitor comprises dasatinib, ibrutinib, acalabrutinib, or zanubrutinib.

13. The method of claim 10, further comprising disposing T lymphocytes selected to express CD16 in combination with Vδ2 into the cell culture media.

14. The method of claim 13, wherein the population of lymphocytes is selected from one or more individuals identified as having at least 35% CD16 positive cells within the population of Vδ2 T lymphocytes obtained from the individual.

15. A cell culture media for expanding T lymphocytes that express CD16 in combination with Vδ2, the media comprising: a bisphosphonate, a cytokine, and optionally a GSK-3β inhibitor.

16. The cell culture media of claim 15, wherein the cell culture media comprises Zoledronate, IL-2, IL-7, IL-15, and/or IL-21.

17. The cell culture media of claim 15, wherein the cell culture media is a serum free cell culture media.

18. The cell culture media of claim 15, wherein the cell culture media contains a tyrosine kinase inhibitor.

19. The cell culture media of claim 15, further comprising a population of T lymphocytes is selected from one or more individuals identified as having at least 35% CD16 positive cells within the population of Vδ2 T lymphocytes obtained from the individual.

20. The cell culture media of claim 19 wherein the population of T lymphocytes has been enriched using an antibody that specifically binds CD16, an antibody that specifically binds a Vδ2 TCR; magnetic bead sorting; and/or fluorescent activated cell sorting (FACS).

21. The cell culture media of claim 19. wherein the population of T lymphocytes has been modified to modulate the expression of one or more endogenous genes and/or the population of cell expresses one or more exogenous transgenes.
